(19) United States
(12) Patent Application Publication
(10) Pub. No.: US 2021/0309742 A1
(43) Pub. Date: Oct. 7, 2021

(54) CONDITIONALLY ACTIVE PROTEINS WITH pH SELECTIVITY

(71) Applicant: BioAtla, Inc., San Diego, CA (US)

(72) Inventor: JAY M. Short, Del Mar, CA (US)

(73) Assignee: BioAtla, Inc., San Diego, CA (US)

(21) Appl. No.: 17/264,702

(22) PCT Filed: Aug. 20, 2019
(86) PCT No.: PCT/US2019/047165

§ 371 Date: Jan. 29, 2021

Related U.S. Application Data
(60) Provisional application No. 62/720,570, filed on Aug. 21, 2018.

(51) Int. Cl. C07K 16/28; G01N 33/68

(52) U.S. Cl. CPC C07K 16/283; G01N 33/6803; C07K 2317/94; C07K 2317/92; C07K 2317/56

(57) ABSTRACT

A method of producing a conditionally active polypeptide from a parent polypeptide, which comprises steps of: (i) evolving the parent polypeptide by introducing mutations into the parent polypeptide to produce mutant polypeptides that have a pI the same as or lower than a pI of the parent polypeptide; (ii) subjecting the mutant polypeptides to a first assay under a normal physiological condition to measure the activity of the mutant polypeptides under the normal physiological condition and a second assay under an aberrant condition to measure the activity of the mutant polypeptides under the aberrant condition, wherein the normal physiological condition and the aberrant condition are the same condition but having different values; and (iii) selecting the conditionally active polypeptide from the mutant polypeptides which exhibits an increased activity in the second assay compared to the same activity in the first assay. Conditionally active polypeptides and uses are also provided.

### FIELD OF THE DISCLOSURE

This disclosure relates to the field of polypeptides with conditional activity. Specifically, this disclosure relates to conditionally active polypeptides which have a pH dependent activity and to a method of generating the conditionally active polypeptides from a parent polypeptide.

### BACKGROUND OF THE DISCLOSURE

There is a considerable body of literature describing methods of evolving proteins for a variety of characteristics, especially enzymes or antibodies, to be active or stable at different conditions. For example, enzymes have been evolved to be stable at higher temperatures. In situations where there is an enzymatic activity improvement at the higher temperature, a substantial portion of the improvement can be attributed to the higher kinetic activity commonly described by the Q10 rule where it is estimated that in the case of an enzyme the turnover doubles for every increase of 10° C.

In addition, there exist natural mutations that destabilize proteins at their normal operating conditions, thus reducing the protein activity at the normal operating conditions. For instance, there are known temperature mutants that are active at a lower temperature, but typically at a reduced level compared to the wild type proteins from which they are derived.

Evolving a parent polypeptide to be inactive or virtually inactive (less than 50%, 30%, or 10% activity and especially 1% activity) at its usual operating condition, while maintaining activity equivalent or better than its activity at aberrant conditions, may require that the destabilizing mutation(s) co-exist with activity increasing mutation(s) that do not counter the destabilizing effect. It is expected that the destabilizing mutation(s) would reduce the polypeptide's activity greater than the effects predicted by standard rules such as Q10, therefore the ability to evolve polypeptides that work efficiently at aberrant conditions, for example, while being less active or inactivated under their normal operating condition, creates conditionally active polypeptides.

It is desirable to generate polypeptides that are conditionally active, for example, less active or virtually inactive at one condition and active at another condition. It is also desirable to generate polypeptides that are activated or inactivated in certain environments, or that are activated or inactivated over time. Besides temperature, other conditions under which the polypeptides can be evolved or improved for conditional activity include pH, osmotic pressure, osmolality, oxidative stress; and electrolyte concentration. In addition to activity of polypeptides, it is often desirable to improve other properties during evolution include chemical resistance, and proteolytic resistance.

Further, it has been observed that the isoelectric point (pI) of a polypeptide is inversely correlated with the half-life of the polypeptide (t_{1/2}), see Momany et al., “Relationship between in vivo degradative rates and isoelectric points of proteins,” PNAS, vol., 73, pp. 3093-3097, 1976. This is especially true for antibodies, because the half-life of antibodies in plasma has been positively correlated with their isoelectric point values. Particularly, antibodies with higher pI values not only have faster systemic clearance, but also lower bioavailability compared with antibodies having a lower pI value. A decrease in the pI of an antibody by 1-2 units has been shown to correlate with a decrease in plasma clearance (i.e., a longer half-life). See Ryman, CPT Pharmacometrics Syst. Pharmacol., vol. 6, pp. 576-588, 2017.

Strategies for changing the pI of polypeptides have been described. For example, U.S. Pat. No. 9,908,932 discloses a method of shifting the isoelectric profile of a recombinant protein having seven protein subpopulations with pis between about 5.45 and about 6.55. The method includes the steps of: (a) culturing mammalian cells comprising a nucleic acid encoding the recombinant protein in a production bioreactor under conditions sufficient to produce a product for a first period of time; and (b) incubating the product under conditions sufficient to shift the isoelectric profile of the product toward a more acidic profile for a second period of time that consists of at least 6 hours. The product with the shifted isoelectric profile shows an increase in the quantity of the fourth and fifth most acidic protein subpopulations of the seven protein subpopulations, and a decrease in the quantity of the first and second most basic protein subpopulations of the seven protein subpopulations.

U.S. Pat. No. 9,605,061 discloses a method for modifying the pI of an antibody by introducing at least 6 amino acid mutations, including substitutions with non-native amino acids in a constant domain selected from one or both of the heavy chain constant domain and light chain constant domain. The substituting amino acids have a pI lower than the native amino acid, such that the pI of the mutant antibody is lowered by at least 0.5 logs relative to the pI of the parent antibody.

US 2009/0324589 discloses a method for increasing the half-life of an IgG antibody in blood by controlling the surface charge of the antibody through modification of residues exposed on the surface of the antibody, including residues in the variable regions. The method comprises the steps of (a) modifying a nucleic acid encoding a parent polypeptide comprising an FcRn-binding domain to change the charge of at least one amino acid residue that is exposed on the surface of the parent polypeptide; (b) culturing a host cell to express the modified nucleic acid to express mutant polypeptides; and (c) collecting the expressed mutant polypeptides comprising an FcRn-binding domain from the host cell culture.

There remains a need for conditionally active polypeptides having a higher activity and/or selectivity in particular environments and/or under particular conditions, and preferably also having an increased half-life in the plasma. The conditionally active polypeptides that also have an increased half-life will not only preferentially act at locations where aberrant conditions are present, such as a tumor microenvironment, but also provide extended action or an increase in overall activity because of their increased half-life. In addition, these conditionally active polypeptides will potentially cause less harmful side effects to normal tissues/organs where normal physiological conditions are present. The potential to reduce side effects allows more prolonged treatments with, or higher doses of, the conditionally active polypeptides leading to a higher efficacy.

WO 2010/104821 and WO 2011/009058 disclose methods for evolving and screening for conditionally active proteins.

### SUMMARY OF THE DISCLOSURE

In one aspect, the disclosure relates to a method of producing a conditionally active polypeptide from a parent polypeptide, comprising steps of (i) evolving the parent polypeptide by introducing one or more mutations into the parent polypeptide to produce one or more mutant polypeptides that have a pI that is the same as or lower than a pI of the parent polypeptide, (ii) subjecting the one or more mutant polypeptides to a first assay under a normal physiological condition to measure the activity of the one or more mutant polypeptides under the normal physiological condition and a second assay under an aberrant condition to measure the activity of the one or more mutant polypeptides under the aberrant condition, wherein the normal physiological condition and the aberrant condition are the same condition but having different values, and (iii) selecting the conditionally active polypeptide from the one or more mutant polypeptides which exhibits an increased activity in the second assay compared to the same activity in the first assay.

In the previous embodiment, the conditionally active polypeptide may have a pI that is lower than the pI of the parent polypeptide.

In any one of the previous embodiments, the conditionally active polypeptide may have a pI below 7.4, or a pI below 7.3, or a pI below 7.2, or a pI below 7.1 or a pI below 7.0.

In any one of the previous embodiments, the one or more mutations may comprise at least one amino acid substitution of a residue of an amino acid for a residue of an amino acid in the parent polypeptide that has a higher pI than a pI of the amino acid that is substituted into the parent polypeptide.

In any one of the previous embodiments, the one or more mutations may comprise 2, 3, 4, 5, 6, 7, 8, 9, or 10 of said substitutions.

In any one of the previous embodiments, the one or more mutations may comprise at least one insertion of a residue of an amino acid that has a lower pI than the pI of the parent polypeptide.

In any one of the previous embodiments, the one or more mutations may comprise 2, 3, 4, or 5 of said insertions.

In any one of the previous embodiments, the one or more mutations may comprise at least one deletion of a residue of an amino acid that has a higher pI than the pI of the parent polypeptide.

In any one of the previous embodiments, the one or more mutations may comprise 2, 3, 4, or 5 of said deletions.

In any one of the previous embodiments, the one or more of the mutations may be located in a position exposed on the surface of the mutant polypeptide.

In any one of the previous embodiments, the evolving step may comprise introduction of one or more additional mutations into the mutant polypeptide.

Any one of the previous embodiments may further comprise a step before step (ii) for confirming that at least 50%, or at least 60%, or at least 70%, or at least 80%, or at least 90%, or at least 95%, or at least 98% of the mutant polypeptides have a pI that is the same as or lower than the pI of the parent polypeptide.

The previous embodiment may further comprise a step of discarding mutant polypeptides that have a pI greater than the pI of the parent polypeptide prior to step (ii).

Any one of the previous embodiments may further comprise a step of measuring a pI of the conditionally active polypeptide.

In any one of the previous embodiments, the pI of the conditionally active polypeptide may be lower than the pI of the parent polypeptide by at least 0.1, or at least 0.2, or at least 0.3, or at least 0.4, or at least 0.5, or at least 0.6, or at least 0.8, or at least 1.0, or at least 1.2, or at least 1.4, or at least 1.5, or at least 1.7, or at least 2.0, or at least 2.5, or at least 3.0, or at least 3.5, or at least 4.0, or at least 5.0 units.

In any one of the previous embodiments, the parent polypeptide may be selected from antibodies, enzymes, hormones, growth factors, cytokines, regulatory proteins, functional peptides, biosimilars, immunomodulators, receptors, and ligands.

In any one of the previous embodiments, the parent polypeptide may be an antibody selected from a full-length antibody, a single chain antibody, an antibody fragment, a heavy chain, a light chain, an Fab, and Fc domain.

In any one of the previous embodiments, the parent polypeptide may be a therapeutic antibody or a candidate antibody being developed for therapeutic use.

In any one of the previous embodiments, the parent polypeptide may be an IgG antibody.

In any one of the previous embodiments, the one or more mutations may be in a variable region of the IgG antibody.

In any one of the previous embodiments, the one or more mutations may be in a constant region of the IgG antibody.

In any one of the previous embodiments, the one or more mutations may be in one or more complementarity determining regions of the IgG antibody.

In any one of the previous embodiments, the condition may be selected from pH, temperature, osmotic pressure, osmolality, oxidative stress, and electrolyte concentration.

In any one of the previous embodiments, the condition may be pH.

In the previous embodiment, the pH of the first assay may be greater than 7.2 to less than 7.6 and the pH of the second assay is less than 7.2 or greater than 7.6.

In any one of the previous embodiments, a ratio of the activity of the conditionally active polypeptide in the second assay to the same activity in the first assay may be at least 1.3, or 1.5, or at least 1.7, or at least 2.0, or at least 3.0, or at least 4.0, or at least 6.0, or at least 8.0, or at least 10.0, or at least 20.0, or at least 40.0, or at least 60.0, or at least 100.0.

In any one of the previous embodiments, both the first assay and second assay may be performed in the presence of a molecule or ion having a molecular weight of less than 900 a.m.u., less than 500 a.m.u., less than 200 a.m.u., or less than 100 a.m.u.

In the previous embodiment, the molecule or ion may be selected from histidine, histamine, hydrogenated adenosine diphosphate, hydrogenated adenosine triphosphate, citrate, bicarbonate, acetate, lactate, bisulfide, hydrogen sulfide, ammonium, dihydrogen phosphate and any combination thereof.

In the previous embodiment, the molecule or ion may be bicarbonate ion having a concentration in a range of from about 3 mM to about 200 mM, from about 5 mM to about 150 mM, from about 5 mM to about 100 mM, from about 10 mM to about 100 mM, from about 20 mM to about 100 mM, from about 25 mM to about 100 mM, from about 30 mM to about 100 mM, from about 35 mM to about 100 mM, from about 40 mM to about 100 mM, or from about 50 mM to about 100 mM.

In the previous embodiment, the molecule or ion may be bisulfide ion having a concentration in a range of from 1 mM to 100 mM, from 2nM to 500 nM, from 3 nM to 200 nM, from 5 nM to 100 nM.

In any one of the previous embodiments, the molecule or ion may be selected from sodium bicarbonate, potassium bicarbonate, sodium bisulfide, or potassium bisulfide.

In any one of the previous embodiments, the physiological condition may be a normal physiological pH and the aberrant condition is an aberrant pH different from the normal physiological pH, and the molecule or ion has a pKa between the normal physiological pH and the aberrant pH.

In the previous embodiment, the pKa of the molecule or ion may be up to 0.1, 0.2, 0.3, 0.4, 0.5, 0.6, 0.8, or 1.0, 2.0 units away from the aberrant pH.

In a second aspect, the disclosure relates to a conditionally active polypeptide derived from a parent polypeptide having a pI, said conditionally active polypeptide having: (a) a pI that is the same as or lower than the pI of the parent polypeptide, and (b) a ratio of at least 1.3 of an activity in a second assay at an aberrant condition to the same activity in a first assay at a normal physiological condition, wherein the activity of the conditionally active polypeptide is measured in the presence of at least one molecule or ion having a molecular weight of less than 900 a.m.u.

In the second aspect, the molecular weight may be less than 500 a.m.u., less than 200 a.m.u., or less than 100 a.m.u.

In any one of the previous embodiments, the ratio of the activity in the second assay at an aberrant condition to the same activity in the first assay at the normal physiological condition may be at least 1.5, or at least 1.7, or at least 2.0, or at least 3.0, or at least 4.0, or at least 6.0, or at least 8.0, or at least 10.0, or at least 20.0, or at least 40.0, or at least 60.0, or at least 100.0.

In any one of the previous embodiments, the pI of the conditionally active polypeptide may be lower than the pI of the parent polypeptide by at least 0.1, or at least 0.2, or at least 0.3, or at least 0.4, or at least 0.5, or at least 0.6, or at least 0.8, or at least 1.0, or at least 1.2, or at least 1.4, or at least 1.5, or at least 1.7, or at least 2.0, or at least 2.5, or at least 3.0, or at least 3.5, or at least 4.0, or at least 5.0 units.

In any one of the previous embodiments, the conditionally active polypeptide may be selected from antibodies, enzymes, hormones, growth factors, cytokines, regulatory proteins, functional peptides, biosimilars, immunomodulators, receptors, and ligands.

In any one of the previous embodiments, the conditionally active polypeptide may be an antibody selected from a full-length antibody, a single chain antibody, an antibody fragment, a heavy chain, a light chain, an Fab, and Fc domain.

In the previous embodiment, the antibody may be an IgG antibody.

In any one of the previous embodiments, the condition may be selected from pH, temperature, osmotic pressure, osmolality, oxidative stress, and electrolyte concentration.

In any one of the previous embodiments, the normal physiological condition may be a pH in a range of from greater than 7.2 to less than 7.6, and the aberrant condition is a pH in a range of 5.5 to less than 7.2.

In the previous embodiment, the molecule or ion may have a pKa between the normal physiological pH and the aberrant pH.

In the previous embodiment, the pKa of the molecule or ion may be up to 0.1, 0.2, 0.3, 0.4, 0.5, 0.6, 0.8, or 1.0, 2.0 units away from the aberrant pH.

In any one of the previous embodiments, the molecule or ion may be selected from histidine, histamine, hydrogenated adenosine diphosphate, hydrogenated adenosine triphosphate, citrate, bicarbonate, acetate, lactate, bisulfide, hydrogen sulfide, ammonium, dihydrogen phosphate and any combination thereof.

In any one of the previous embodiments, the molecule or ion may be bicarbonate ion having a concentration in a range of from about 3 mM to about 200 mM, from about 5 mM to about 150 mM, from about 5 mM to about 100 mM, from about 10 mM to about 100 mM, from about 20 mM to about 100 mM, from about 25 mM to about 100 mM, from about 30 mM to about 100 mM, from about 35 mM to about 100 mM, from about 40 mM to about 100 mM, or from about 50 mM to about 100 mM.

In any one of the previous embodiments, the molecule or ion may be bisulfide ion having a concentration in a range of from 1 mM to 100 mM, from 2nM to 500 nM, from 3 nM to 200 nM, from 5 nM to 100 nM.

In any one of the previous embodiments, the molecule or ion may be selected from sodium bicarbonate, potassium bicarbonate, sodium bisulfide, or potassium bisulfide.

In yet another aspect, the disclosure relates to a pharmaceutical composition comprising an effective amount of the conditionally active polypeptide and a pharmaceutically acceptable carrier.

In yet another aspect, the disclosure relates to a use of the conditionally active polypeptide for treatment of solid tumors, inflamed joints, or brain diseases or disorders.

In yet another aspect, the disclosure relates to a method of treatment of solid tumors, inflamed joints, or brain diseases or disorders comprising a step of administering the conditionally active polypeptide to a patient in need of said treatment.

### BRIEF DESCRIPTION OF THE DRAWING

FIG. 1 is a diagram showing the formation of salt bridges in deoxyhemoglobin, where three amino acid residues form two salt bridges that stabilize the T quaternary structure of the deoxyhemoglobin, leading to lower affinity to oxygen.

### DEFINITIONS

In order to facilitate understanding of the examples provided herein, certain frequently occurring methods and/or terms will be defined herein. Definitions for the following terms are incorporated by reference in their entirety from U.S. Pat. No. 8,709,755 B2: “agent”, “ambiguous base requirement”, “amino acid”, “amplification”, “chimeric property”, “cognate”, “comparison window”, “conservative amino acid substitutions”, “corresponds to”, “degrading effective”, “defined sequence framework”, “defined sequence kemal”, “digestion”, “directional ligation”, “DNA shuffling”, “drug” or “drug molecule”, “effective amount”, “epitope”, “enzyme”, “evolution” or “evolving”, “fragment” or “derivative” or “analog”, “full range of single amino acid substitutions”, “gene”, “genetic instability”, “heterologous”, “homologous” or “homeologous”, “industrial applications”, “identical” or “identity”, “areas of identity”, “isolated”, “isolated nucleic acid”, “ligand”, “ligation”, “linker” or “spacer”, “microenvironment”, “molecular property to be evolved”, “mutations”, “N,N,G/T”, “normal physiological conditions” or “wild type operating conditions”, “nucleic acid molecule”, “nucleic acid molecule”, “nucleic acid sequence coding for” or a “DNA coding sequence of” or a “nucleotide sequence encoding”, “nucleic acid encoding an enzyme (protein)” or “DNA encoding an enzyme (protein)” or “polynucleotide encoding an enzyme (protein)”, “specific nucleic acid molecule species”, “assembling a working nucleic acid sample into a nucleic acid library”, “nucleic acid library”, “construct”, “oligonucleotide” (or synonymously an “oligo”), “homologous”, “operably linked”, “parental polynucleotide set”, “patient” or “subject”, “physiological conditions”, “population”, “pro-form”, “pseudorandom”, “quasi-repeated units”, “random peptide library”, “random peptide sequence”, “receptor”, “recombinant” enzymes, “synthetic” enzymes, “related polynucleotides”, “reductive reassortment”, “reference sequence”, “repetitive Index (RI)”, “restriction site”, “selectable polynucleotide”, “sequence identity”, “similarity”, “specifically bind”, “specific hybridization”, “specific polynucleotide”, “stringent hybridization conditions”, “substantially identical”, “substantially pure enzyme”, “substantially pure”, “treating”, “variable segment”, and “variant”.

Definitions for the following terms are incorporated by reference in their entirety from WO 2017/078839: “about,” “activity,” “antibody,” “antibody-dependent cell-mediated cytotoxicity” or “ADCC,” “antigen” or “Ag,” “antisense RNA,” “biosimilar” or “follow-on biologic,” “biosimilar antibody,” “cancer” or “cancerous,” “chimeric antigen receptor” or “CAR” or “CARs,” “cytokine” or “cytokines,” “electrolyte,” “full length antibody,” “growth factor,” “hormones,” “immunomodulator,” “individual” or “subject,” “library,” “ligand,” “receptor,” “microRNA” or “miRNA,” “multispecific antibody,” “nanoparticle,” “naturally-occurring,” “recombinant antibody,” “regulatory protein,” “small interfering RNA” or “siRNA,” “therapeutic protein,” “therapeutically effective amount,” “tumor microenvironment,” “wild-type.”

The term “amino acid residues exposed on the surface” of a polypeptide as used herein refers to an amino acid residue of the polypeptide, which, when the polypeptide is present in a liquid (e.g. blood, human serum, cytoplasm, etc.), has at least a portion of its side chain in contact with the liquid. The amino acid residues exposed on the surface of the polypeptide may be determined by X-ray crystallography. The amino acid residues that may be exposed on the surface can also be determined, for example, using coordinates from a three-dimensional model of an antibody using a computer program such as InsightII program (Accelrys). Other software available for such purposes includes, for example, the SYBYL Biopolymer Module software (Tripos Associates). When an algorithm requires a user input size parameter, the “size” of a probe used in the calculation may be set to about 1.4 Angstrom (Å) or less in radius. For example, methods for determining surface-exposed amino acid residues and regions using software for personal computers have been described by Pacios (Pacios, Comput. Chem, vol. 18, pp. 377-386, 1994; J. Mol. Model., vol. 1, pp. 46-53, 1995).

The term “conditionally active polypeptide” refers to a variant or mutant of a parent polypeptide which is more active than the parent polypeptide under at least one condition (e.g., aberrant condition) and less active than the parent polypeptide under a second condition (e.g., normal physiological condition), or refers to a variant or mutant of a parent polypeptide which is more active under at least one condition (e.g., aberrant condition) that under a second condition (e.g., normal physiological condition). In one embodiment, the conditionally active polypeptide is at least 1.3, or at least 1.5, or at least 2.0, or at least 2.5, or at least 3.0, or at least 5.0, or at least 10, or at least 15, or at least 20, or at least 40, or at least 60, or at least 80, or at least 100 times more active under the second, aberrant condition than under the first, normal physiological condition. This conditionally active polypeptide may exhibit activity in one or more selected locations of the body and/or exhibit increased or decreased activity at another location in the body. For example, in one aspect, the conditionally active polypeptide is virtually inactive at body temperature, but is active at lower temperatures. Conditionally active polypeptides include conditionally active proteins, protein fragments, antibodies, antibody fragments, enzymes, enzyme fragments, receptors and fragments of receptors cytokines and fragments thereof, hormones and fragments thereof, ligands and fragments thereof, regulatory proteins and fragments thereof, growth factors and fragments thereof., as well as proteins including a stress protein, a vault-related protein, a neuron protein, a digestive tract protein, a growth factor, a mitochondrial protein, a cytosolic protein, an animal protein, a structural protein, a plant protein and fragments of any of these proteins. Each of the conditionally active polypeptides described herein is preferably a conditionally active biologic polypeptide.

The term “increase of the half-life in plasma” or “prolongation of the half-life in plasma” for a polypeptide as used herein refers increased retention time of the polypeptide in plasma (half-life in plasma (t_{1/2})), or decreased clearance (CL) in plasma. This can be represented as the area under the concentration curve (AUC) over time.

The term “isoelectric point” or “pI” of a polypeptide as used herein refers to the pH at which the polypeptide carries no net electrical charge. The pI of the polypeptide can be determined experimentally, or calculated based on the amino acid sequence of the polypeptide. For example, the pI can be determined by isoelectric focusing electrophoresis of the polypeptide, which is known to those skilled in the art. The theoretical calculation of pI can be determined using amino acid sequence analysis software (GENETYX and the like) based on the amino acid of the polypeptide.

The term “isoelectric point variant” or “pI variant” of a parent polypeptide refers to a mutant polypeptide of the parent polypeptide that has a decreased pI in comparison with the parent polypeptide from which the mutant polypeptide was derived either via substituting a residue of an amino acid having a higher pI with a residue of an amino acid having a lower pI, deleting a residue of an amino acid having a higher pI than the pI of the parent polypeptide, or inserting a residue of an amino acid with a lower pI than the pI of the parent polypeptide. The present invention extends to both conditionally active pI variants as well as to conditionally active polypeptides having the same pI as the parent polypeptide.

The terms “parent polypeptide” and “parent protein” as used herein refer to a polypeptide or protein that may be evolved to produce a conditionally active polypeptide using the methods described herein. The parent polypeptide may be a non-naturally occurring protein. For example, a therapeutic polypeptide or protein or a mutant or variant polypeptide may be used as a parent polypeptide. Examples of parent polypeptides include antibodies, antibody fragments, enzymes, enzyme fragments cytokines and fragments thereof, hormones and fragments thereof, ligands and fragments thereof, receptors and fragments thereof, regulatory proteins and fragments thereof, and growth factors and fragments thereof.

The term “pH-dependent” as used herein refers to a polypeptide having a property or activity that is different at different pH values.

The term “polypeptide” as used herein refers to an amino acid polymer in which the amino acids are joined together through peptide or disulfide bonds. A polypeptide may be a full-length naturally-occurring amino acid chain or a fragment, mutant or variant thereof, such as a selected region of the amino acid chain that is of interest in a binding interaction. A polypeptide may also be a synthetic amino acid chain, or a combination of a naturally-occurring amino acid chain or fragment thereof and a synthetic amino acid chain. A fragment refers to an amino acid sequence that is a portion of a full-length protein, and will be typically between about 8 and about 500 amino acids in length, preferably about 8 to about 300 amino acids, more preferably about 8 to about 200 amino acids, and even more preferably about 10 to about 50 or 100 amino acids in length. Additionally, amino acids other than naturally-occurring amino acids, for example 0-alanine, phenyl glycine and homoarginine, may be included in the polypeptides. Commonly-encountered amino acids which are not gene-encoded may also be included in the polypeptides. The amino acids may be either the D- or L- optical isomer. The D-isomers are preferred for use in a specific context, further described below. In addition, other peptidomimetics are also useful, e.g. in linker sequences of polypeptides (see Spatola, 1983, in Chemistry and Biochemistry of Amino Acids. Peptides and Proteins, Weinstein, ed., Marcel Dekker, New York, p. 267). In general, the term “protein” is not intended to convey any significant difference from the term “polypeptide” other than to include structures which comprise two or several polypeptide chains held together by covalent or non-covalent bonds.

The term, “small molecule” refers to molecules that typically have a molecular weight of less than 900 a.m.u., or more preferably less than 500 a.m.u. or more preferably less than 200 a.m.u. or even more preferably less than 100 a.m.u. The same molecular weight ranges apply to ions used in these assays. In the assays and environments of the present invention, small molecules or ions may often be present as a mixture of a molecule and a deprotonated ion of the molecule, depending primarily on the pH of the assay or environment.

### DETAILED DESCRIPTION

The present disclosure provides a method for generating a conditionally active polypeptide from a parent polypeptide. The method comprises the steps of (i) evolving the parent polypeptide by introducing one or more mutations into the parent polypeptide to produce one or more mutant polypeptides that have a pI that is the same as or lower than the pI of the parent polypeptide; (ii) subjecting the one or more mutant polypeptides to a first assay under a normal physiological condition to measure the activity of the one or more mutant polypeptides under the normal physiological condition and a second assay under an aberrant condition to measure the activity of the one or more mutant polypeptides under the aberrant condition, wherein the normal physiological condition and the aberrant condition are the same condition but having different values; and (iii) selecting the conditionally active polypeptide from the one or more mutant polypeptides which exhibits an increased activity in the second assay at the aberrant condition compared to the same activity in the first assay at the normal physiological condition.

### A. Mutations Influencing the pI of the Polypeptide

A parent polypeptide may be mutated to generate a conditionally active polypeptide having the same or a decreased pI in comparison with the pI of the parent polypeptide. Preferably, the conditionally active polypeptide will have a decreased pI in comparison with the pI of the parent polypeptide. Alternatively, or at the same time, the conditionally active polypeptide may have a pI below 7.4, or below 7.3, or below 7.2, or below 7.1, or below 7.0. This conditionally active polypeptide will also exhibit pH selectivity, i.e. a higher activity at the pH at which activity is desired than at, for example, a normal physiological pH which may be in a range from greater than 7.2 to less than 7.6.

Any suitable mutagenesis technique may be employed, including amino acid residue substitutions, deletions, insertions and combinations thereof. The amino acid residue substitutions replace a native amino acid residue in the parent polypeptide with a residue of another amino acid having a lower pI than the pI of the amino acid of the replaced native amino acid residue. The pI's of amino acids are shown in Table 1. Although this table shows the pI of amino acids as individual molecules rather than the amino acid residues located in a polypeptide, the effect of the pI of the amino acid residues when part of a polypeptide follows the trend for the pI of the amino acids in Table 1. Thus, for example, replacing a native residue of an amino acid with a higher pI with a residue of an amino acid having a pI lower than the higher pI of the native amino acid, will lower the pI of the polypeptide. In practice, a native amino acid residue in the parent polypeptide may be substituted with a residue of another amino acid that has a pI lower than the pI of the native amino acid. For example, if the native amino acid residue is a residue of a basic amino acid, it may be substituted with a residue of a weak acidic amino acid or a residue of a strong acidic amino acid in order to lower the pI of the polypeptide. In another example, if the native amino acid residue is a residue of a weak acidic amino acid, it may be substituted with a residue of a strong acidic amino to lower the pI of the polypeptide.

TABLE 1
pI's of Amino Acids

| | Amino Acid | pI |
|---|---|---|
| Basic amino | Arginine | 10.76 |
| acids | Lysine | 9.74 |
| | Histidine | 7.59 |
| Neutral or | Proline | 6.30 |
| weak acidic | Isoleucine | 6.02 |
| amino acids | Alanine | 6.00 |
| | Leucine | 5.98 |
| | Glycine | 5.97 |
| | Valine | 5.96 |
| | Tryptophan | 5.89 |
| | Methionine | 5.74 |
| | Serine | 5.68 |
| | Tyrosine | 5.66 |
| | Glutamine | 5.65 |
| | Threonine | 5.60 |
| | Phenylalanine | 5.48 |
| | Asparagine | 5.41 |
| | Cysteine | 5.07 |
| Strong acidic | Glutamic acid | 3.22 |
| amino acids | Aspartic acid | 2.77 |

In some embodiments, two or more amino acid residues are substituted into the parent polypeptide to influence the pI of the parent polypeptide. For example, two, three, four, five, six, seven, eight, nine, ten or more amino acid residue substitutions may be introduced into the parent polypeptide. In some embodiments, each substitution substitutes a residue of an amino acid with a lower pI for a residue of an amino acid with a higher pI. In other embodiments, only a portion of the substitutions substitute a residue of an amino acid with a lower pI for a residue of an amino acid with a higher pI. In the latter case, the combination of substitutions is selected such that the overall effect of the multiple substitutions will be to either maintain the same pI for the mutant polypeptide as the pI of the parent polypeptide or to provide a lower pI for the mutant polypeptide than the pI of the parent polypeptide.

In some embodiments, the amino acid residue substitutions used to influence the pI may be conservative substitutions, non-conservative substitutions, or a combination thereof.

Amino acid residue deletions that decrease the pI of the parent polypeptide include deleting a residue of an amino acid that has a pI that is higher than the pI of the parent polypeptide. For example, when the pI of the parent polypeptide is about 7.2, deletion of any one or more residues of basic amino acids from the parent polypeptide will decrease the pI of the parent polypeptide. In some embodiments, at least two, three, four, five or more amino acid residues are deleted from the parent polypeptide. In some embodiments, each deletion deletes a residue of an amino acid with a lower pI than the pI of the parent polypeptide. In other embodiments, only a portion of the deletions delete a residue of an amino acid with a higher pI than the pI of the parent polypeptide. In the latter case, the combination of deletions is selected such that the overall effect of the multiple deletions will be to maintain or lower the pI of the polypeptide.

Amino acid residue insertions that decrease the pI of the parent polypeptide include inserting a residue of an amino acid that has a pI that is lower than the pI of the parent polypeptide. For example, when the pI of the parent polypeptide is about 7.2, insertion of any one or more residues of the weak acidic amino acids or strong acidic amino acids into the parent polypeptide will result in decrease of the pI of the parent polypeptide. In some embodiments, at least two, three, four, five or more amino acid residues are inserted into the parent polypeptide. In some embodiments, each insertion inserts a residue of an amino acid with a lower pI than the pI of the parent polypeptide. In other embodiments, only a portion of the insertions insert a residue of an amino acid with a lower pI than the pI of the parent polypeptide. In the latter case, the combination of insertions is selected such that the overall effect of the multiple insertions will be to maintain or lower the pI of the polypeptide.

In some embodiments, combinations of two or more of the above-described amino acid residue substitutions, amino acid residue deletions, and amino acid residue insertions is used to influence the pI of the parent polypeptide. For example, the mutant polypeptides may include one or more amino acid residue substitutions and one or more amino acid residue deletions. In another example, the mutant polypeptides may have one or more amino acid residue substitutions and one or more amino acid residue insertions. In yet another example, the mutant polypeptides may have one or more amino acid residue deletions and one or more amino acid residue insertions. In a still further example, the mutant polypeptides may have all of one or more amino acid residue substitutions, one or more amino acid residue deletions, and one or more amino acid residue insertions. In each case, all or only a portion of the insertions, substitutions and/or deletions may employ residues of amino acids having a pI lower than either the pI of the polypeptide or the pI of the amino acid that is being substituted. In each case, the combination of insertions, substitutions and/or deletions is selected such that the overall effect of the multiple insertions, substitutions and/or deletions will be to maintain or lower the pI of the polypeptide.

The desired mutant polypeptides will have a pI that is the same as or lower than the pI of the parent polypeptide. Some mutant polypeptides will have a pI that is the same as the pI of the parent polypeptide. Other mutant polypeptides may have a pI that is at least 0.1, or at least 0.2, or at least 0.3, or at least 0.4, or at least 0.5, or at least 0.6, or at least 0.8, or at least 1.0, or at least 1.2, or at least 1.4, or at least 1.5, or at least 1.7, or at least 2.0, or at least 2.5, or at least 3.0, or at least 3.5, or at least 4.0, or at least 5.0 units lower than the pI of the parent polypeptide.

In some embodiments, the native amino acid residues being substituted, the amino acid residues being deleted, and/or the positions where the amino acid residue is inserted are exposed on the surface of the parent polypeptide. It is understood that the mutations at the exposed amino acid residues or positions have a lower probability of disrupting the three-dimensional structure of the parent polypeptide.

The amino acid residue substitutions, insertions and deletions can be carried out on a nucleotide sequence encoding the parent polypeptide, for example, by site-directed mutagenesis (Kunkel et al., Proc. Natl. Acad. Sci. USA 82:488-492 (1985)) or overlap extension PCR. When the parent polypeptide is an antibody, the mutations may also be achieved by affinity maturation of the antibody, or by chain shuffling of antibody heavy or light chains; or by antigen panning-based selection using phage-display libraries (Smith et al., Methods Enzymol. 217:228-257 (1993)). These mutagenesis methods can be performed alone or in appropriate combinations.

In some further embodiments, in addition to mutations that maintain or decrease the pI of relative to the pI of the parent polypeptide, the mutant polypeptide may also be subjected to additional mutagenesis at positions other than the positions having the mutations introduced to maintain or lower the pI. The additional mutagenesis may employ any known mutagenesis methods, for example, Comprehensive Positional Evolution, Comprehensive Positional Deletion, Comprehensive Positional Insertion, or combinations thereof, which have been described in detail in US 2013/0116125.

For example, a parent polypeptide may be mutated to include one or more amino acid residue substitutions that maintain or decrease the pI relative to the pI of the parent polypeptide. For example, the mutated polypeptide may also be subjected to Comprehensive Positional Evolution including, for example, comprehensive positional substitution, comprehensive positional deletion and comprehensive positional insertion at positions other than the positions having the one or more mutations already introduced to lower the pI of the polypeptide.

The pI of any polypeptide may be determined by isoelectric focusing gel electrophoresis (e.g., capillary isoelectric focusing gel electrophoresis) or other methods (see, e.g., the methods described in Righetti et al., Methods Biochem. Anal. 54:379-409, 2011; Friedman et al., Methods Enzymol. 463:515-540, 2009; Koshel et al., Proteomics 12:2918-2926, 2012; Sommer et al., Electrophoresis 30:742-757, 2009; Shimura et al., Electrophoresis 30:11-28, 2009). Further, there are methods that can estimate or calculate the pI of a polypeptide based on its amino acid sequence, such as the methods described in Ribeiro J M. Sillero A., Computers in Biology & Medicine 20(4):235-42, 1990; Ribeiro J M. Sillero A., Computers in Biology & Medicine 21(3):131-41, 1991; and Sillero A. Ribeiro J M., Analytical Biochemistry 179(2):319-25, 1989.

Any proteins, including antibodies, enzymes, hormones, growth factors, cytokines, regulatory proteins, functional peptides, biosimilars, immunomodulators, therapeutic proteins, receptors, and ligands may be used as the parent polypeptide of the present invention. The parent polypeptide may also be a fragment of any of the above proteins. For example, the parent polypeptide may be a tissue plasminogen activator, streptokinase, urokinase, renin, hyaluronidase, calcitonin gene-related peptide (CGRP), substance P (SP), neuropeptide Y (NPY), vasoactive intestinal peptide (VTP), vasopressin or angiostatin. For another example, the parent polypeptide may be a stress protein, a vault-related protein, a neuron protein, a digestive tract protein, a growth factor, a mitochondrial protein, a cytosolic protein, an animal protein, a structural protein, a plant protein, or a fragment of any of these proteins.

In one exemplary embodiment, the parent polypeptide is an antibody. The parent antibody may be a therapeutic antibody, or a candidate antibody being developed for therapeutic use. Examples of parent antibodies include rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT/US2003/040426, entitled “Immunoglobulin Variants and Uses Thereof”). A number of antibodies that target members of the family of epidermal growth factor receptors, including EGFR (ErbB-1), Her2/neu (ErbB-2), Her3 (ErbB-3), Her4 (ErbB-4), may benefit from pI engineered constant region(s) of the invention. For example the pI engineered constant region(s) of the invention may find use in an antibody that is substantially similar to trastuzumab (Herceptin®, Genentech) (see for example U.S. Pat. No. 5,677,171), a humanized anti-Her2/neu antibody approved to treat breast cancer; pertuzumab (rhuMab-2C4, Omnitarg™), currently being developed by Genentech; an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Pat. Application Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80); TheraClM hR3 (YM Biosciences, Canada and Centro de Immunologia Molecular, Cuba (U.S. Pat. No. 5,891,996; U.S. Pat. No. 6,506,883; Mateo et al, 1997, Immunotechnology, 3(1):71-81); mAb-806 (Ludwig Institute for Cancer Research, Memorial Sloan-Kettering) (Jungbluth et al. 2003, Proc Natl Acad Sci USA. 100(2):639-44); KSB-102 (KS Biomedix); MR1-1 (WAX, National Cancer Institute) (PCT WO 0162931A2); and SC100 (Scancell) (PCT WO 01/88138). In another preferred embodiment, the pI engineered constant region(s) of the present invention may find use in alemtuzumab (Campath®, Millenium), a humanized monoclonal antibody currently approved for treatment of B-cell chronic lymphocytic leukemia. The engineered constant region(s) of the present invention may find use in a variety of antibodies that are substantially similar to other clinical products and candidates, including but not limited to muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen, abciximab (ReoPro®), developed by Centocor/Lilly, basiliximab (Simulect®), developed by Novartis, palivizumab (Synagis®), developed by Medlmmune, infliximab (Remicade®), an anti-TNFalpha antibody developed by Centocor, adalimumab (Humira®), an anti-TNFalpha antibody developed by Abbott, Humicade™, an anti-TNFalpha antibody developed by Celltech, etanercept (Enbrel®), an anti-TNFalpha Fc fusion developed by Immunex/Amgen, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 In development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-132 antibody being developed by Cambridge Antibody Technology, J695, an anti-IL-12 antibody being developed by Cambridge Antibody Technology and Abbott, CAT-192, an anti-TGF131 antibody being developed by Cambridge Antibody Technology and Genzyme, CAT-213, an anti-Eotaxinl antibody being developed by Cambridge Antibody Technology, LymphoStat-B™ an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc., Avastin™ (bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair™ (Omalizumab), an anti-IgE antibody being developed by Genentech, Raptiva™ (Efalizumab), an anti-CD11 a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD4OL antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide™ (labetuzumab), an anti-carcinoembryonic antigen (CEA) antibody being developed by Immunomedics, LymphoCideTM (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being devel-oped by Medarex, MDX-070 being developed by Medarex, MDX-018 being developed by Medarex, Osidem™ (IDM-1), and anti-Her2 antibody being developed by Medarex and Immuno-Designed Molecules, HuMax™-CD4, an anti-CD4 antibody being developed by Medarex and Genmab, HuMax-IL15, an anti-IL15 antibody being developed by Medarex and Genmab, CNTO 148, an anti-TNFa antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF™, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, and MLN01, an anti-Beta2 integrin antibody being developed by Xoma, an pI-ADC antibody being developed by Seattle Genetics, all of the above-cited references in this paragraph are expressly incorporated herein by reference.

The parent antibody may be a full-length antibody, an antibody fragment, a single chain antibody, an Fab or Fe domain. The parent antibody may be an IgG antibody. Of the several antibody isotypes, the IgG antibody has a sufficiently large molecular weight, and thus its major metabolic pathway is not through renal excretion. The IgG antibody is known to be recycled through a salvage pathway via FeRit, and thus has a long in vivo half-life. The IgG antibody is assumed to be mainly metabolized via a metabolic pathway in endothelial cells (He et al., J. immunol., vol. 160, pp. 10294035, 1998). Specifically, it is believed that when the IgG antibodies are taken up into endothelial cells nonspecifically, the IgG antibodies are recycled by binding to FeRti, while the IgG antibodies that do not bind to FcRn are degraded. The plasma half-life of an IgG antibody is inversely related with its pI, as described in, e.g., WO2007/114319 and WO2009/041643.

Among the IgG antibodies, the parent antibody may be an IgG1 antibody which is a common isotype for therapeutic antibodies for a variety of reasons, including high effector function. However, the heavy constant region of IgG1 has a higher pi than that of IgG2 (8.10 versus 7.31). By introducing some IgG2 amino acid residues at particular positions into the IgG1 backbone by amino acid residue substitutions, the pI of the resulting IgG1 is lowered, and additionally exhibits longer half-life in plasma than the parent IgG1 antibody. For example, IgG1 has a glycine at position 137, and IgG2 has a glutamic acid (strong acidic amino acid) at the same position. Substituting the glycine at position 137 of IgG1 with glutamic acid will decrease the pI of the mutant IgG1 antibody, which will increase the half-life of the IgG1 antibody.

In some embodiments, one or more of the mutations or combinations of mutations described herein for maintaining or decreasing the pI relative to the pI of the parent polypeptide are located in the antibody heavy chain, the antibody light chain or in both the antibody heavy chain and the antibody light chain.

### B. Mutations in the Heavy Chain of an Antibody

In some embodiments, the mutation or combination of mutations described herein for the purpose of maintaining or decreasing the pI of the polypeptide are made in at least in the CH1 region of the heavy chain of an IgG antibody. In these embodiments, the mutations can be independently selected from mutations at positions 119, 131, 133, 137, 138, 164, 192, 193, 196, 199, 203, 205, 208, 210, 214, 217 and 219 of the CH1 region and any combination of mutations at these positions. Mutations can be introduced by substitution, deletion or insertion at one of these 17 positions or at all possible combinations and sub-combinations of these 17 positions. For example, a mutant antibody may have 1, 2, 3, 4, 5, 6, 7, 8, 9, 10, 11, 12, 13, 14, 15, 16, or 17 CH1 substitutions at one or more of these 17 positions. Thus, any single mutation or combination of mutations in the CH1 region are possible. Further, the one or more mutations in the CH1 region may optionally be combined with one or more other mutation or combinations of mutations in any of the CH2, CH3, hinge and LC regions. In such case, the combination of mutations in the CH1, CH2, CH3, hinge and LC regions may be selected to reduce the pI of the mutant antibody relative to the pI of the parent antibody.

Useful substitutions that may decrease the pI of the heavy chain include substitution of an aspartic acid or glutamic acid residue at one or more of positions 121, 124, 129, 132, 134, 126, 152, 155, 157, 159, 101, 161, 162, 165, 176, 177, 178, 190, 191, 194, 195, 197, 212, 216 and 218 in the CH1 region of an IgG antibody heavy chain.

Specific substitutions in the CH1 region of the antibody heavy chain may include, but are not limited to, a non-native glutamic acid at position 119, a non-native cysteine at position 131, a non-native arginine, lysine or glutamine at position 133, a non-native glutamic acid at position 137, a non-native serine at position 138, a non-native glutamic acid at position 164, a non-native asparagine at position 192; a non-native phenylalanine at position 193, a non-native lysine at position 196, a non-native threonine at position 199, a non-native aspartic acid at position 203, a non-native glutamic acid or glutamine at position 205, a non-native aspartic acid at position 208, a non-native glutamic acid or glutamine at position 210, a non-native threonine at position 214, a non-native arginine at position 217 and a non-native cysteine at position 219, and any combination thereof.

In some embodiments, the mutations are made in the hinge region of the antibody heavy chain, including at positions 221, 222, 223, 224, 225, 233, 234, 235 and 236. Specifically, 1, 2, 3, 4 or 5 mutations and particularly, 1, 2, 3, 4 or 5 substitutions can be made at positions 221-225. Again, all possible combinations are contemplated, alone or with other mutations in other regions.

Specific mutations in the hinge region of the antibody heavy chain may include, but are not limited to, a deletion at position 221, a non-native valine or threonine at position 222, a deletion at position 223, a non-native glutamic acid at position 224, a deletion at position 225, a deletion at position 235, a deletion or a non-native alanine at position 236 and any combination thereof. In some cases, only one or more these mutations are introduced in the hinge region of the antibody heavy chain, and in other embodiments, one or more of these mutations are combined with one or more of the mutations in other regions of the parent antibody in any combination.

In some embodiments, the mutations can be made in the CH2 region of the antibody heavy chain, including at positions 274, 296, 300, 309, 320, 322, 326, 327, 334 and 339. Specifically, 1, 2, 3, 4, 5, 6, 7, 8, 9 or 10 mutations can be made in any combination in these 10 positions of the CH2 region of the antibody heavy chain.

Specific substitutions in the CH2 region of the antibody heavy chain may include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and any combination of these substitutions, as well as in combination with one or more other mutations in other regions of the antibody.

The mutations can be independently selected from mutations at positions 355, 359, 362, 384, 389, 392, 397, 418, 419, 444 and 447 of the CH3 region of the antibody heavy chain. The mutant antibody may have 1, 2, 3, 4, 5, 6, 7, 8, 9, 10 or 11 mutations at these locations in the CH3 region in any combination. In addition, as is described herein, any one or more mutations in the CH3 region can be combined with one or more other mutations in the CH2, CH1, hinge and LC regions of the antibody.

Specific substitutions in the CH3 region of the antibody heavy chain may include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and non-native aspartic acid at position 447 and any combination of two or more of these substitutions.

Thus, all possible combinations of the following antibody heavy chain deletions or substitutions can be made, with each mutation being optionally included or excluded: a non-native glutamic acid at position 119, a non-native cysteine at position 131, a non-native arginine, lysine or glutamine at position 133, a non-native glutamic acid at position 137, a non-native serine at position 138, a non-native glutamic acid at position 164, a non-native asparagine at position 192, a non-native phenylalanine at position 193, a non-native lysine at position 196, a non-native threonine at position 199, a non-native aspartic acid at position 203, a non-native glutamic acid or glutamine at position 205, a non-native aspartic acid at position 208, a non-native glutamic acid or glutamine at position 210, a non-native threonine at position 214, a non-native arginine at position 217 and a non-native cysteine at position 219, a deletion at position 221, a non-native valine or threonine at position 222, a deletion at position 223, a non-native glutamic acid at position 224, a deletion at position 225, a deletion at position 235, a deletion at position 221, a non-native valine or threonine at position 222, a deletion at position 223, a non-native glutamic acid at position 224, a deletion at position 225, and a deletion at position 235, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a non-native aspartic acid at position 447.

### C. Mutations in the Antibody Light Chain

In some embodiments, the mutations may be in the light chain of an IgG antibody. The mutations can be located at positions independently selected from positions 126, 145, 152, 156, 169, 199, 202 and 207 of the light chain. The antibody may have 1, 2, 3, 4, 5, 6, 7 or 8 mutations in the light chain at these positions in any combination. In addition, any single or combination of light chain mutations can be combined with any one or more of the above-described antibody heavy chain mutations.

Specific substitutions in the antibody light chain may include, but are not limited to, a non-native glutamine or glutamic acid at position 126, a non-native glutamine, glutamic acid or threonine at position 145; a non-native aspartic acid at position 152, a non-native glutamic acid at position 156, a non-native glutamine or glutamic acid at position 169, a non-native glutamic acid at position 199, a non-native glutamic acid at position 202 and a non-native glutamic acid at position 207.

### D. Heavy and Light Chain Mutations

In some embodiments, a mutant antibody may have mutations in both the heavy and light chains as described above. In some embodiments, the mutant antibody may have mutations only in the heavy chain, in which case the mutant antibody will have the light chain of the parent antibody. In some embodiments, a mutant antibody may have mutations in only the light chain, in which case the mutant antibody will have the heavy chain of the parent antibody.

Thus, any possible combination of the following mutations in the antibody heavy and light chains can be made, with each mutation being optionally included or excluded: a) heavy chain: a non-native glutamic acid at position 119; a non-native cysteine at position 131; a non-native arginine, lysine or glutamine at position 133; a non-native glutamic acid at position 137; a non-native serine at position 138; a non-native glutamic acid at position 164; a non-native asparagine at position 192; a non-native phenylalanine at position 193, a non-native lysine at position 196, a non-native threonine at position 199, a non-native aspartic acid at position 203, a non-native glutamic acid or glutamine at position 205, a non-native aspartic acid at position 208, a non-native glutamic acid or glutamine at position 210, a non-native threonine at position 214, a non-native arginine at position 217 and a non-native cysteine at position 219, a deletion at position 221, a non-native valine or threonine at position 222, a deletion at position 223, a non-native glutamic acid at position 224, a deletion at position 225, a deletion at position 235, a deletion at position 221, a non-native valine or threonine at position 222, a deletion at position 223, a non-native glutamic acid at position 224, a deletion at position 225, and a deletion at position 235, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447; and light chain: b) a non-native glutamine or glutamic acid at position 126, a non-native glutamine, glutamic acid or threonine at position 145; a non-native aspartic acid at position 152, a non-native glutamic acid at position 156, a non-native glutamine or glutamic acid at position 169, a non-native glutamic acid at position 199, a non-native glutamic acid at position 202 and a non-native glutamic acid at position 207.

### E. Mutations in the Constant Regions of the Antibody

In some embodiments, the one or more mutations may be located in the heavy chain constant region, the light chain constant region, or both, of an antibody, such as an IgG antibody. For example, there may be 1, 2, 3, 4, 5, 6, 7, 8, 9, or 10 mutations in the heavy chain constant region and/or the light chain constant region of the antibody.

The mutations in the heavy chain constant region and/or light chain constant region may be sufficient to decrease the pI of the parent antibody by at least 0.2, 0.4, 0.6, 08, 1.0, 1.2, 1.4, 1.6, 1.8, 2.0, 2.5, 3.0, 3.5, 4.0, 4.5 or 5.0 units, in comparison with pI of the parent antibody.

### F. Variable Regions of the Parent Antibody

In some embodiments, the one or more mutations are located in the heavy chain variable region, the light chain variable region, or both, of an antibody, such as an IgG antibody. In one embodiment, the mutations are located in one or more of the complementarity determining regions (CDRs), for example, one or more of CDR1, CDR2, and CDR3, and/or the framework regions (FRs), for example, one or more of FR1, FR2, FR3, and FR4 of the heavy chain and/or the light chain of the antibody. Introducing mutations into the variable regions may provide advantage in comparison with mutations in the constant regions, because mutations in the constant regions may potentially lead to increased immunogenicity.

In one example, the mutations located are in positions within the variable regions that are not masked by antigen binding. Positions that are not masked by antigen binding are the positions that remain exposed on the surface of the antigen-bound antibody. Alternatively, the mutations may be made at locations that do not substantially interfere with antigen binding.

Methods for identifying CDRs and FRs are known (Kabat et al., Sequence of Proteins of Immunological Interest (1987), National Institute of Health, Bethesda, Md.; Chothia et al.; Nature, vol. 342, p. 877, 1989). Modifiable amino acid residues in the FRs include residues that directly bind to an antigen via non-covalent bonds (Amit et al., Science, 233:747-53, 1986), residues that have some impact or effect on the CDR structure (Chothia et al., J. Mol. Biol., 196:901-917, 1987), and residues involved in the interaction between heavy chain variable region and light chain variable region (Patent Publication EP 239400A1).

The mutagenesis methods described above produce a set of one or more mutant polypeptides. These mutant polypeptides may have a pI that is the same as or lower than the pI of the parent polypeptide. Some mutant polypeptides that can be generated using various mutagenesis methods described above, may have a pI that is the same as or greater than the pI of the parent polypeptide. This may be caused, for example, by additional mutations introduced by Comprehensive Positional Evolution, Comprehensive Positional Deletion, Comprehensive Positional Insertion, or combinations thereof, which may overcome the effect of the mutations introduced for the purpose of maintaining or decreasing pI. For this reason, in some embodiments, the present invention confirms that a particular mutant polypeptide or at least a substantial portion of the mutant polypeptides have a pI that is lower than the pI of the parent polypeptide. This may be accomplished by isoelectric focusing gel electrophoresis that demonstrates that a substantial portion of the mutant polypeptides are focused at a region having a lower pH than the pI of the parent polypeptide. For example, at least 50%, or at least 60%, or at least 70%, or at least 80%, or at least 90%, or at least 95%, or at least 98% of the mutant polypeptides may have a pI that is lower than the pI of the parent polypeptide.

In some embodiments, the present invention optionally filters the mutant polypeptides to exclude some or all of the mutant polypeptides that have a pI that is the same as or higher than the pI of the parent polypeptide. The filtering of these mutant polypeptides may also be accomplished by isoelectric focusing gel electrophoresis whereby the mutant polypeptides focused at a region having a lower pH than the pI of the parent polypeptide may be collected and used for further process. The mutant polypeptides focused at a region having the same or higher pH than the pI of the parent polypeptide may be excluded.

### G. Screening for Conditionally Active Polypeptides

The mutant polypeptides, are screened for conditional activity. In this manner, polypeptides can be identified that have both conditional activity and a lower pI than the parent polypeptide. This is a desirable combination of features, particularly for antibodies such as therapeutic antibodies.

The methods of screening the mutant polypeptides for selection of the conditionally active polypeptide have been described in WO 2017/078839. The conditionally active polypeptide may be selected by screening for an increased activity at an aberrant condition that deviates from a normal physiological condition, compared to the same activity of the same mutant polypeptide at the corresponding normal physiological condition, and optionally,

(a) screening for an increased activity at an aberrant condition that deviates from a normal physiological condition, compared to the same activity of the parent polypeptide at the same aberrant condition,

(b) screening for a decreased activity at a normal physiological condition compared with the same activity of the parent polypeptide at the same normal physiological condition, or

(c) a combination of (a) and (b) above.

The conditionally active polypeptides may have a selectivity, i.e. a ratio of the activity at the aberrant condition to the activity at the normal physiological condition, of at least about 1.3, or at least about 1.5, or at least about 1.7, or at least about 2.0, or at least about 3.0, or at least about 4.0, or at least about 6.0, or at least about 8.0, or at least about 10.0, or at least about 20.0, or at least about 40.0, or at least about 60.0, or at least about 100.0.

The aberrant condition and the normal physiological condition are different values of the same condition. For example, the aberrant and normal physiological conditions may be two different temperatures, or two different pH values. The conditions may be selected from temperature, pH, osmotic pressure, osmolality, oxidative stress, electrolyte concentration, as well as combinations of two or more such conditions.

For example, the normal physiological condition for temperature may be a normal human body temperature of 37.0° C., while the aberrant condition for temperature may be a temperature different from the temperature of 37.0° C., such as a temperature in tumor microenvironment which may be 1-2° C. higher than the normal physiological temperature. In another example, the normal physiological condition may be a normal human physiological pH in the range of 7.2-7.8, or 7.2-7.6 and an aberrant pH such as in the range of 5.5-7.2, 6-7, or 6.2-6.8 as may be found in a tumor microenvironment.

The assays under both normal physiological condition and aberrant condition may be performed in an assay media. The assay media may be a solution, which may contain, for example, a buffer as well as other components. Common buffers that can be used in the assay media include citrate buffers such as sodium citrate, phosphate buffers, bicarbonate buffers such as the Krebs buffer, phosphate buffered saline (PBS) buffer, Hank's buffer, Tris buffer, HEPES buffer, etc. Other buffers known to a person skilled in the art to be suitable for the assays may be used. These buffers may be used to mimic a characteristic or component of the composition of a bodily fluid, of a human or animal such as blood plasma or lymphatic fluid.

The assay solutions useful in the methods of the invention may contain at least one component selected from inorganic compounds, ions and organic molecules, preferably ones that are commonly found in a bodily fluid of a mammal such as a human or animal. Examples of such components include nutritional components and metabolites, as well as any other components that may be found in a bodily fluid. The present invention contemplates that this component may or may not be part of the buffer system. For example, the assay solutions may be PBS buffer with added bicarbonate where bicarbonate is not part of PBS buffer. Alternatively, bicarbonate is a component in Krebs buffer.

The component may be present in both assay solutions (for the first and second conditions) at substantially the same concentration, while the two assay solutions different in other aspect such as pH, temperature, electrolyte concentrations, or osmotic pressure. Thus, the component is used as a constant, rather than the difference between the two conditions of the first and second conditions, or the normal physiological condition and aberrant condition, respectively.

In some embodiments, the component is present in both assay solutions at a concentration that is close to or the same as the normal physiological concentration of the component is mammals, especially in humans.

The inorganic compounds or ions may be selected from one or more of boric acid, calcium chloride, calcium nitrate, di-ammonium phosphate, magnesium sulfate, mono-ammonium phosphate, mono-potassium phosphate, potassium chloride, potassium sulfate, copper sulfate, iron sulfate, manganese sulfate, zinc sulfate, magnesium sulfate, calcium nitrate, chelates of calcium, copper, iron, manganese and zinc, ammonium molybdate, ammonium sulphate, calcium carbonate, magnesium phosphate, sodium bisulfide, potassium bisulfide, sodium bicarbonate, potassium bicarbonate, potassium nitrate, hydrochloric acid, carbon dioxide, sulfuric acid, phosphoric acid, carbonic acid, uric acid, hydrogen chloride, urea, phosphorus ion, sulfuric ion, chloride ion, magnesium ion, sodium ion, potassium ion, ammonium ion, iron ion, zinc ion and copper ion.

Examples of normal physiological concentrations of some of the inorganic compounds include: uric acid in a concentration range of 2-7.0 mg/dL, calcium ion in a concentration range of 8.2-11.6 mg/dL, chloride ion in a concentration range of 355-381 mg/dL, iron ion in a concentration range of 0.028-0.210 mg/dL, potassium ion in a concentration range of 12.1-25.4 mg/dL, sodium ion in a concentration range of 300-330 mg/dL, carbonic acid in a concentration range of 15-30 mM, citrate ion at about 80 μM, histidine ion in the range of 0.05-2.6 mM, histamine in the range of 0.3-1 μM, HAPT ion (hydrogenated adenosine triphosphate) in the range of 1-20 μM, and HADP ion in the range of 1-20 μM.

In some embodiments, the ion present in the assay solutions for both the first condition and second condition, or the normal physiological condition and aberrant condition, respectively, is selected from hydroxide ion, halide ion (chloride, bromide, iodide), oxyhalide ion, sulfate ion, magnesium ion, calcium ion, bisulfate ion, carbonate ion, bicarbonate ion, sulfonate ion, oxyhalide ion, nitrate ion, nitrite ion, phosphate ion, hydrogen phosphate ion, dihydrogen phosphate ion, persulfate ion, monopersulfate ion, borate ion, ammonium ion, or organic ion, such as carboxylate ion, phenolate ion, sulfonate ion (organosulfate such as methyl sulfate), vanadate ion, tungstate ion, borate ion, organoboronate ion, citrate ion, oxalate ion, acetate ion, pentaborate ion, histidine ion, and phenolate ion.

The organic compounds present in the assay solutions for both the first condition and second condition, or the normal physiological condition and aberrant condition, respectively, may be selected from, for example, amino acids such as Histidine, Alanine, Isoleucine, Arginine, Leucine, Asparagine, Lysine, Aspartic acid, Methionine, Cysteine, Phenylalanine, Glutamic acid, Threonine, Glutamine, Tryptophan, Glycine, Valine, Pyrrolysine, Proline, Selenocysteine, Serine, Tyrosine and mixtures thereof.

Examples of a normal physiological concentration of some of the amino acids include: Alanine at 3.97 ±0.70 mg/dL, Arginine at 2.34±0.62 mg/dL, Glutamic acid at 3.41±1.39 mg/dL, Glutamine at 5.78±1.55 mg/dL, Glycine at 1. 77±0.26 mg/dL, Histidine at 1.42±0.18 mg/dL, Isoleucine at 1.60±0.31 mg/dL, Leucine at 1.91±0.34 mg/dL, Lysine at 2.95±0.42 mg/dL, Methionine at 0.85±0.46 mg/dL, Phenylalanine at 1.38±0.32 mg/dL, Threonine at 2.02±6.45 mg/dL, Tryptophan at 1.08±0.21 mg/dL, Tyrosine at 1.48±0.37 mg/dL and Valine at 2.83±0.34 mg/dL.

The organic compounds present in the assay solutions for both the first condition and second condition, or the normal physiological condition and aberrant condition, respectively, may be selected from non-protein nitrogen-containing compounds such as creatine, creatinine, guanidino acetic acid, uric acid, allantoin, adenosine, urea, ammonia and choline. Examples of normal physiological concentrations of some of these compounds include: creatine at 1.07 ±0.76 mg/dL, creatinine at from 0.9 to 1.65 mg/dL, guanidino acetic acid at 0.26±0.24 mg/dL, uric acid at 4.0±2.9 mg/dL, allantoin at from 0.3 to 0.6 mg/dL, adenosine at 1.09±0.385 mg/dL, urea 27.1±4.5 mg/dL and choline at from 0.3 to 1.5 mg/dL.

The organic compounds present in the assay solutions for both the normal physiological condition and aberrant condition may be selected from organic acids such as citric acid, α-ketoglutaric acid, succinic acid, malic acid, fumaric acid, acetoacetic acid, β-hydroxybutyric acid, lactic acid, pyruvic acid, α-ketonic acid, acetic acid, and volatile fatty acids. Examples of normal physiological concentrations of some of these organic acids include: citric acid at 2.5±1.9 mg/dL, α-ketoglutaric acid at 0.8 mg/dL, succinic acid at 0.5 mg/dL, malic acid at 0.46±0.24 mg/dL, acetoacetic acid at from 0.8 to 2.8 mg/dL, β-hydroxybutyric acid at 0.5±0.3 mg/dL, lactic acid at from 8 to 17 mg/dL, pyruvic acid at 1.0±0.77 mg/dL, a-ketonic acids at from 0.6 to 2.1 mg/dL, volatile fatty acids at 1.8 mg/dL.

The organic compounds present in the assay solutions for both the normal physiological condition and aberrant condition, may be selected from sugars (carbohydrates) such as glucose, pentose, hexose, xylose, ribose, mannose and galactose, as well as disaccharides including lactose, GlcNAcβ1-3Gal, Galα1-4Gal, Manal-2Man, GalNAcβ1-3Gal and O—, N—, C—, or S-glycosides. Examples of normal physiological concentrations of some of these sugars include: glucose at 83±4 mg/dL, polysaccharides at 102±73 mg/dL (as hexose), glucosamine at 77±63 mg/dL, hexuronates at from 0.4 to 1.4 mg/dL (as glucuronic acid) and pentose at 2 .55±0.37 mg/dL.

The organic compounds present in the assay solutions for both the normal physiological condition and aberrant condition, may be selected from fats or their derivatives such as cholesterol, lecithin, cephalin, sphingomyelin and bile acid. Examples of normal physiological concentrations of some of these compounds include: free cholesterol at from 40 to 70 mg/dL, lecithin at from 100 to 200 mg/dL, cephalin at from 0 to 30 mg/dL, sphingomyelin at from 10 to 30 mg/dL and bile acids at from 02. To 0.3 mg/dL (as cholic acid).

The organic compounds present in the assay solutions for both the normal physiological condition and aberrant condition, may be selected from proteins such as fibrinogen, antihaemophilic globulin, immune y-globulin, immune euglobulins, isoagglutinins, β-pseudoglobulin, glycoproteins, lipoproteins and albumin. For example, the normal physiological concentration of mammal serum albumin is 3.5-5.0 g/dL. In one embodiment, the albumin is bovine serum albumin.

The organic compounds present in the assay solutions for both the normal physiological condition and aberrant condition, may be selected from vitamins such as Vitamin A, Carotene, Vitamin E, Ascorbic acid, Thiamine, Inositol, Folic acid, Biotin, Pantothenic acid, Riboflavin. Examples of normal physiological concentrations of some of these vitamins include: Vitamin A at from 0.019 to 0.036 mg/dL, Vitamin E at from 0.90 to 1.59 mg/dL, Inositol at from 0.42 to 0.76 mg/dL, Folic acid at from 0.00162 to 0.00195 mg/dL and biotin from 0.00095 to 0.00166 mg/dL.

The concentration of the inorganic compound, ion, or organic molecule in the assay solutions (for both assays under the normal physiological condition and aberrant condition) may be within the normal range of physiological concentration of the inorganic compound, ion, or organic molecule in human or animal blood serum. However, the concentrations outside of the normal physiological range may also be used. For example, the normal range in human serum for magnesium ion is 1.7-2.2 mg/dL, and calcium is 8.5 to 10.2 mg/dL. The concentration for magnesium ion in the assay solutions may be from about 0.17 mg/dL to about 11 mg/dL. The concentration for calcium ion in the assay solutions may be from about 0.85 mg/dL to about 51 mg/dL. As a general rule, the concentration of the inorganic compound, ion, or organic molecule in the assay solutions may be as low as 5%, or 10%, or 20%, or 30%, or 40%, or 50%, or 60%, or 70%, or 80% of the normal physiological concentration of the inorganic compound, ion, or organic molecule in human serum, or as high as 1.5 times, or 2 times, or 3 times, or 4 times or 5 times, or 7 times or 9 times or 10 times or even 20 times the normal physiological concentration of the inorganic compound, ion, or organic molecule in human serum. Different components of the assay solutions may be used at different concentration levels relative to their respective normal physiological concentrations.

The assays under the normal physiological condition and aberrant condition are used to measure the activity of the mutant polypeptides. During the assays both the mutant polypeptide and its binding partner are present in the assay solutions. The relationship between the mutant polypeptide and its binding partner may be, for example, antibody-antigen, ligand-receptor, enzyme-substrate, or hormone-receptor. In order for a mutant polypeptide to manifest its activity, the mutant polypeptide should be able to come into contact with and bind to its binding partner. The activity of the mutant polypeptide on its binding partner is then manifested and measured after the binding between the mutant polypeptide and its binding partner.

In some embodiments, the ions used in the assay may function in forming a bridge between the mutant polypeptide being screened and its binding partner, particularly those including charged amino acid residues. The ion may thus be capable of binding to both the mutant polypeptide and its binding partner through hydrogen bonds and/or ionic bonds. This may assist the binding between the mutant polypeptide and its binding partner by allowing the ion to reach a site that may be hard to reach by a large molecule (mutant polypeptide or its binding partner). In some cases, the ion in the assay solutions may increase the probability of the mutant polypeptide and its binding partner binding to one another. Further, the ion may additionally or alternatively assist the binding between the mutant polypeptide and its binding partner by binding to a larger molecule (mutant polypeptide or its binding partner). This binding may alter the conformation of the large molecule and/or cause the larger molecule to remain in a particular conformation that facilitates binding with its binding partner.

It has been observed that the ions can assist the binding between the mutant polypeptide and its binding partner, possibly by forming ionic bonds with the mutant polypeptide and its binding partner. Thus, the screening may be much more efficient and more hits (candidate conditionally active polypeptides) can be identified in comparison with the same assays without the ion. Suitable ions may be selected from magnesium ion, sulfate ion, bisulfate ion, carbonate ion, citrate ion, HAPT ion, HADP ion, bicarbonate ion, nitrate ion, nitrite ion, phosphate ion, hydrogen phosphate ion, dihydrogen phosphate ion, persulfate ion, monopersulfate ion, borate ion, lactate ion, citrate ion, histidine ion, histamine ion, and ammonium ion.

It has been found that the ions function to assist the binding between the mutant polypeptide and its binding partner at a pH near a pKa of the ion. Such ions are preferably relatively small in relation to the size of the mutant polypeptides.

In one embodiment, when the aberrant condition is a pH that is different from the normal physiological pH under the normal physiological condition, the ions suitable for increasing the number of hits for candidate conditionally active polypeptides may be selected from ions having a pKa that is close to the aberrant pH to be tested in the assay. For example, the pKa of the ion may be up to 2 pH units away from the aberrant pH, up to 1 pH unit away from the aberrant pH, up to 0.8 pH unit away from the aberrant pH, up to 0.6 pH unit away from the aberrant pH, up to 0.5 pH unit away from the aberrant pH, up to 0.4 pH unit away from the aberrant pH, up to 0.3 pH unit away from the aberrant pH, up to 0.2 pH unit away from the aberrant pH, or up to 0.1 pH unit away from the aberrant pH.

Exemplary pKa's of ions useful in the present invention, which pKa's may vary slightly at different temperatures, are as follows: ammonium ion having a pKa at about 9.24, dihydrogen phosphate having a pKa at about at 7.2, acetic acid having a pKa at about 4.76, histidine having a pKa at about 6.04, bicarbonate ion having a pKa at about 6.4, citrate having a pKa at 6.4, lactate ion having a pKa at about 3.86, histamine having a pKa at about 6.9, HATP having a pKa at 6.95 (HATP³⁻⇔ATP⁴⁻+H⁺) and HADP having a pKa at 6.88 (HADP³⁻⇔ADP⁴⁻+H⁺).

In one embodiment, the conditionally active polypeptides are assayed and selected in the presence bisulfide. Bisulfide has a pKa of 7.05. In some embodiments, different concentrations of bisulfide may be used in the assays representing the normal and aberrant physiological conditions. Alternatively, the assay media for both the normal physiological condition and aberrant condition have approximately the same concentration of bisulfide and also some difference in the value of the particular condition, for example, the assay may be conducted at different pH's. The concentration of bisulfide to be used in the assay may be from 1 mM to 100 mM. Preferably, the assay medium has a bisulfide concentration of from 2 to 500 nM, or from 3 to 200 nM, or from 5 to 100 nM. In some aspect, the bisulfide concentration may be from 1 mM to 20 mM, or from 2 mM to 10 mM. Assays conducted in the presence of bisulfide are known.

In certain embodiments, once the pH for the aberrant condition (i.e., aberrant pH) is known, the ion suitable for increasing the hits for candidate conditionally active polypeptides may be selected from ions that have a pKa that is at or near the aberrant pH, for example, the candidate ions may have a pKa up to 4 pH units away from the aberrant pH, up to 3 pH unites away from the aberrant pH, up to 2 pH units away from the aberrant pH, up to 1 pH unit away from the aberrant pH, up to 0.8 pH unit away from the aberrant pH, up to 0.6 pH unit away from the aberrant pH, up to 0.5 pH unit away from the aberrant pH, up to 0.4 pH unit away from the aberrant pH, up to 0.3 pH unit away from the aberrant pH, up to 0.2 pH unit away from the aberrant pH, or up to 0.1 pH unit away from the aberrant pH.

As stated above, the ion is most effective at assisting the binding between the mutant polypeptide and its binding partner at a pH that is at or close to the pKa of the ion. For example, it has been found that in an assay solution with a pH 7.2-7.6, the bicarbonate ion (having pKa about 6.4) is not very effective in assisting the binding between the mutant polypeptide and its binding partner. As the pH in the assay solution decreased to 6.7 and further to around 6.0, the bicarbonate ion became increasingly effective in assisting the binding between the mutant polypeptide and its binding partner. As a result, more hits could be identified in the assay at pH 6.0 in comparison with assay at pH 7.2-7.6. Similarly, histidine is not very effective in assisting the binding between the mutant polypeptide and its binding partner at pH 7.4. As the pH of the assay solution is decreased to 6.7 and further to around 6.0, histidine becomes increasingly effective in assisting the binding between the mutant polypeptide and its binding partner also allowing more hits to be identified at pHs in a range of about 6.2-6.4, for example.

It has been found that, when the pHs of the assay solutions for the normal physiological condition (i.e., a normal physiological pH) and aberrant condition (i.e., an aberrant pH) are different, an ion with pKa in the range of from about the middle point of the normal physiological pH and the aberrant pH to about the aberrant pH can greatly assist the binding between the mutant polypeptide being screened and its binding partner. As a result, the screening assay is much more efficient in founding more hits or candidate conditionally polypeptides with high activity at the aberrant condition.

In some embodiments, the pKa may even be at least one pH unit away from the aberrant pH. When the aberrant pH is an acidic pH, the pKa of a suitable ion may be in the range of from (aberrant pH−1) to the middle point between the aberrant pH and the normal physiological pH. When the aberrant pH is a basic pH, the pKa of a suitable ion may be in the range of from (aberrant pH+1) to the middle point between the aberrant pH and the normal physiological pH. The ions may be selected from those described in this application. However, many more ions that have not been explicitly described in the application may also be used. It is understood that, once the aberrant pH and the normal physiological pH are selected for the screening assays, a person skilled in the art can use the guiding principles described herein to select any ion with a suitable pKa for increasing the efficiency of screening in identifying more hits with high activity at the aberrant condition.

For example, when the aberrant pH is 8.4 and the normal physiological pH is 7.4 for an exemplary screening, any ion with a pKa in the range of about 7.9 (the middle point) to 9.4 (i.e., 8.4+1) may be used in the screening. Some ions with a pKa in this range include ions derived from tricine (pKa 8.05), hydrazine (pKa 8.1), bicine (pKa 8.26), N-(2-Hydroxyethyl) piperazine-N′-(4-butanesulfonic acid) (pKa 8.3), N-Tris lhydroxymethyllmethyl-3-aminopropanesulfonic acid (pKa 8.4), taurine (pKa 9.06). For another example, when the aberrant pH is 6 and the normal physiological pH is 7.4 for an exemplary screening, any ion with a pKa in the range of about 5 (i.e., 6-1) to 6.7 (the middle point) may be used in the screening. Some ions with a pKa in this range include ions derived from malate (pKa 5.13), pyridine (pKa 5.23), piperazine (pKa 5.33), cacodylate (pKa 6.27), succinate (pKa 5.64), 2-(N-morpholino)ethanesulfonic acid (pKa 6.10), citrate (pKa 6.4), histidine (pKa 6.04) and bis-tris (6.46). A person skilled in the art will be able to consult a vast number of chemical manuals and text books to identify the known chemical compounds that can be converted to ions with a pKa falling in the ranges, including both inorganic chemical compounds and organic chemical compounds. Among the chemical compounds with a suitable pKa, the ones with a smaller molecular weight may be preferred.

Consequently, the present invention unexpectedly found that production of conditionally active polypeptides eventually identified not only depends on generating the right polypeptide mutants, but also depend on using an ion with a suitable pKa in the assay solutions. The invention contemplates that in addition to generating a large library of mutant polypeptides (e.g., through CPE and CPS), efforts should also be made to find a suitable ion (with proper pKa) for use in the assay solutions, because the ion can facilitate efficiently selecting the mutants with high activity from the large library. It is further contemplated that, without the suitable ion, the screening is less efficient and the probability of finding the mutants with high activity is decreased. Consequently, it may require multiple rounds of screening to achieve the same number of mutants with high activity without the suitable ion.

The ion in the assay solutions may be formed in situ from a component of the assay solution or be directly included in the assay solution. For example, CO₂ from the air may dissolve in the assay solution to provide carbonate and bicarbonate ions. For another example, sodium dihydrogen phosphate may be added to the assay solution to provide dihydrogen phosphate ions.

The concentration of this component in the assay solutions (for both assay under the normal physiological condition and assay under the aberrant condition) may be the same or substantially the same as the concentration of the same component that is typically found in a naturally-occurring bodily fluid of a mammal, such as a human. In other embodiments, the concentration of the component may be higher, especially when the component is an ion that can function to assist the binding between the mutant polypeptide and its binding partner, because it has been observed that higher concentration of such ion can form ionic bonds with the mutant polypeptide and its binding partner, practically facilitate the bindings and increase the probability of finding more hits or candidate conditionally active polypeptides.

In some embodiments, the concentration of the ion in the assay solutions may positively correlate with the probability of finding more hits using the assay, particularly when concentrations in excess of normal physiological concentrations are employed. For example, human serum has a concentration of about 15-30 mM of bicarbonate ion. In one example, as the concentration of bicarbonate ion in the assay solutions was increased from 3 mM to 10 mM, to 20 mM, to 30 mM, to 50 mM and to 100 mM, the number of hits in the assay also increased with each increase in bicarbonate concentration. In view of this, the assay solutions may employ concentrations of bicarbonate ranging from about 3 mM to about 200 mM, or from about 5 mM to about 150 mM or from about 5 mM to about 100 mM, or from about 10 mM to about 100 mM or from about 20 mM to about 100 mM or from about 25 mM to about 100 mM or from about 30 mM to about 100 mM or from about 35 mM to about 100 mM or from about 40 mM to about 100 mM or from about 50 mM to about 100 mM.

In another embodiment, the concentration of citrate in the assay solutions may be from about 30 μM to about 120 μM, or from about 40 μM to about 110 μM, or from about 50 μM to about 110 μM, or from about 60 μM to about 100 μM, or from about μM to about 90 μM, or about

In one embodiment, the normal physiological condition is a normal physiological pH in the range of 7.2-7.6 and the aberrant condition is an aberrant pH in the range of 5.5-7.2, 6-7, or 6.2-6.8. The assay solution for the assay under the normal physiological condition has the normal physiological pH and 50 mM of bicarbonate ion. The assay solution for the assay under the aberrant condition has the aberrant pH and 50 mM of bicarbonate ion. Because the pKa of bicarbonate ion is at about 6.4, the bicarbonate ion can assist the binding between the mutant polypeptides and its binding partner at the aberrant pH pf 6.0-6.4, such as pH 6.0 or 6.2.

In yet another embodiment, the normal physiological condition is a normal physiological pH in the range of 7.2-7.6 and the aberrant condition is an aberrant pH in the range of 5.5-7.2, 6-7, or 6.2-6.8. The assay solution for the assay under the normal physiological condition has the normal physiological pH and 80 μM of citrate ion. The assay solution for the assay under the aberrant condition has the aberrant pH and 80 μM of citrate ion. Because the citrate ion has a pKa of 6.4, the citrate ion can effectively assist the binding between the mutant polypeptides and the binding partner in the assay solution for aberrant condition with pH 6.0-6.4. Therefore more candidate conditionally active polypeptides may be identified that have higher binding activity under condition of pH 6.0-6.4 and lower activity under condition of pH at 7.2-7.8. The other ions, including acetate, histidine, bicarbonate, HATP and HADP, function in a similar way to enable an assay solution containing the ion to effectively screening for mutant polypeptides with a higher binding activity at a pH around the pKa of the ion and a lower binding activity at a pH that is different from the pKa of the ion (e.g., normal physiological pH).

In yet another embodiment, the normal physiological condition is a normal physiological temperature at 37° C. and the aberrant condition is an aberrant temperature at 38-39° C. (temperature in some tumor microenvironments). The assay solution for the assay under the normal physiological condition has the normal physiological temperature and 20 mM of bicarbonate ion. The assay solution for the assay under the aberrant condition has the aberrant temperature and 20 mM of bicarbonate ion.

In yet another embodiment, the normal physiological condition is a particular concentration of an electrolyte in normal human serum and the aberrant condition is the concentration of the same electrolyte in a different, aberrant concentration which may be present at a different location in the animal or human or may result from a condition of the animal or human that alters the normal physiological concentration of an electrolyte in human serum.

The binding between a mutant polypeptide and/or its binding partner can also be influenced in a number of other ways. Typically, this influence will be exerted by inclusion of one or more additional components in the assay solutions. These additional components may be designed to interact with either the mutant polypeptide, the binding partner or both. In addition, these additional components may use combinations of two or more interactions as well as combinations of two or more types of interactions to influence the binding.

In one embodiment, the binding interaction of interest is between an antibody and an antigen. In this embodiment, one or more additional components may be included in the assay solutions to exert influence on the antibody, antigen or both. In this manner, the desired binding interaction may be enhanced.

In addition to the ions that can form ionic bonds with a mutant polypeptide and/or its binding partner to assist the binding between the mutant polypeptide and the binding partner, the present invention also includes other components that may be employed assist binding between a mutant polypeptide and its binding partner. In one embodiment, molecules that can form hydrogen bonds with a mutant polypeptide and/or its binding partner are employed. In another embodiment, molecules that are capable of hydrophobic interaction with a mutant polypeptide and/or its binding partner may be used. In yet another embodiment, molecules that are capable of Van der Waals' interactions with a mutant polypeptide and/or its binding partner are contemplated.

The hydrogen bond is a relatively weak, noncovalent interaction between a hydrogen covalently bonded to an electronegative atom, such as carbon, nitrogen, oxygen, sulfur, chlorine, or fluorine (hydrogen bond donor), with an unshared electron pair of an electron donor atom, such as nitrogen, oxygen, sulfur, chlorine, or fluorine (hydrogen bond acceptor).

Components capable of forming a hydrogen bond with a mutant polypeptide and/or its binding partner include organic molecules as well as inorganic molecules with a polar bond. Mutant polypeptides and/or binding partners for mutant polypeptides typically contain amino acids that can form hydrogen bonds. Suitable amino acids have a side chain with a polar group that is capable of forming a hydrogen bond. Non-limiting examples of suitable amino acids include glutamine (Gln), glutamic acid (Glu), arginine (Arg) asparagines (Asn), aspartic acid (Asp), lysine (Lys), histidine (His), serine (Ser), threonine (Thr), tyrosine (Tyr), cysteine (Cys), methionine (Met), and tryptophan (Trp).

These amino acids can function as both hydrogen donors and hydrogen acceptors. For example, the oxygen atom in an —OH group such as may be found in Ser, Thr, and Tyr, the oxygen atom in a —C═O group such as may be found in Glu and Asp, the sulfur atom in an —SH group or —SC— such as may be found in Cys and Met, the nitrogen atom in a —NH₃⁺ group such as may be found in Lys and Arg, and the nitrogen atom in an —NH— group such as may be found in Trp, His and Arg, may all function as a hydrogen acceptor. Also, groups in this list including a hydrogen atom (e.g. —OH, —SH, NH₃⁺ and —NH—) may function as a hydrogen donor.

In some embodiments, the backbone of the mutant polypeptide and/or its binding partner may also participate in forming one or more hydrogen bonds. For example, the backbone may have a repeating structure of —(C═O)—NH— such as in peptide bonds. The oxygen and nitrogen atoms in this structure may function as hydrogen acceptors, while the hydrogen atom may participate in the hydrogen bond.

The inorganic compounds that have at least one polar bond involving a hydrogen or oxygen atom that may be used for hydrogen bonding may include, for example, H₂O, NH₃, H₂O₂, hydrazine, carbonates, sulfates and phosphates. Organic compounds such as alcohols; phenols; thiols; aliphatic, amines, amides; epoxides, carboxylic acids; ketones, aldehydes, ethers, esters, organochlorides, and organofluorides. Compounds that can form hydrogen bonds are well known in in the chemical literature, such as those discussed in, for example, “The Nature of the Chemical Bond,” by Linus Pauling, Cornell University Press, 1940, pages 284 to 334.

In some embodiments, the alcohols may include methanol, ethanol, propanol, isopropanol, butanol, pentanol, 1-hexanol, 2-octanol, 1-decanol, cyclohexanol, and the higher alcohols; diols such as ethylene glycol, propylene glycol, glycerol, diethylene glycol, and polyalkylene glycols. Suitable phenols include hydroquinone, resorcinol, catechol, phenol, o-, m-, and p-cresol, thymol, alpha and beta-naphthol, pyrogallol, guaiacol, and phloroglucinol. Suitable thiols include methanethiol, ethanethiol, 1-propanethiol, 2-propanethiol, butanethiol, tert-butyl mercaptan, pentanethiols, hexanethiol, thiophenol, dimercaptosuccinic acid, 2-mercaptoethanol, and 2-mercaptoindole. Suitable amines include methylamine, ethylamine, propylamine, isopropylamine, aniline, dimethylamine and methylethylamine, trimethylamine, aziridine, piperidine, N-methylpiperidine, benzidine, cyclohexyl amine, ethylene diamine, hexamethylene diamine, o-, m-, and p-toluidine and N-phenylpiperidine. Suitable amides include ethanamide, N,N-dimethylacetamide, N,N-dimethyl formamide, N,N-dimethyl methoxy acetamide and N-methyl-N-p-cyanoethyl formamide The epoxides may include ethylene oxide, propylene oxide, tert-butyl hydroperoxide, styrene oxide, epoxide glycidol, cyclohexene oxide, di-tert-butyl peroxide, cumene hydroperoxide or ethylbenzene hydroperoxide, isobutylene oxide, and 1,2-epoxyoctane. The carboxylic acids may include terephthalic acid, isophthalic acid, phthalic acid, salicylic acid, benzoic acid, acetic acid, lauric acid, adipic acid, lactic acid, citric acid, acrylic acid, glycine, hexa-hydrobenzoic acid, o-, m-, and p-toluic acids, nicotinic acid, isonicotinic acid, and para-aminobenzoic acid. The ketones may include acetone, 3-propanone, butanone, pentanone, methylethyl ketone, diisobutyl ketone, ethyl butyl ketone, methyl isobutyl ketone, methyl tert-butyl ketone, cyclohexanone, acetone, methyl ethyl ketone, methyl propyl ketone, methyl butyl ketone, methyl amyl ketone, methyl hexyl ketone, diethyl ketone, ethyl butyl ketone, dipropyl ketone, diisobutyl ketone, diacetone alcohol, phorone, isophorone, cyclohexanone, methyl cyclohexanone, and acetophenone. The aldehydes may include formaldehyde, acetaldehyde, propionaldehyde, butyraldehyde, benzaldehyde, cinnamaldehyde, sobutyraldehyde, valeraldehyde, octaldehyde, benzaldehyde, cinnamaldehyde, cyclohexanone, salicylaldehyde, and furfural. The esters include ethyl acetate, methyl acetate, ethyl formate, butyl acetate, ethyl lactate, ethyl butyrate, propyl acetate, ethyl formate, propyl formate, butyl formate, amyl formate, methyl acetate, ethyl acetate, propyl acetate, butyl acetate, amyl acetate, methyl isoamyl acetate, methoxybutyl acetate, hexyl acetate, cyclohexyl acetate, benzyl acetate, methyl propionate, ethyl propionate, butyl propionate, amyl propionate, methyl butyrate, ethyl butyrate, butyl butyrate, amyl butyrate, methyl acetoacetate, and ethyl acetoacetate. Ethers that may be used in the present invention include dimethyl ether, methyl ethyl ether, diethyl ether, methyl propyl ether, and dimethoxyethane. The ethers may be cyclic, such as ethylene oxide, tetrahydrofuran, and dioxane.

The organochlorides include chloroform, pentachloroethane, dichloromethane, trichloromethane, carbon tetrachloride, tetrachloromethane, tetrachloroethane, pentachloroethane, trichloroethylene, tetrachloroethylene, and ethylene dichloride. The organofluorides may include fluoromethane, difluoromethane, trifluoromethane, trifluoroethane tetrafluoroethane, pentafluoroethane, difluoropropane, trifluoropropane, tetrafluoropropane, pentafluoropropane, hexafluoropropane, and heptafluoropropane,

Hydrogen bonds may be divided by the strength of the bond: strong, moderate, or weak hydrogen bonds (Jeffrey, George A.; An introduction to hydrogen bonding, Oxford University Press, 1997). The strong hydrogen bonds have donor-acceptor distances of 2.2-2.5 Å and energies in the range of 14-40 kcal/mol. The moderate hydrogen bonds have donor-acceptor distances of 2.5-3.2 Å and energies in the range of 4-15 kcal/mol. The weak hydrogen bonds have donor-acceptor distances of 3.2-4.0 Å and energies in the range of <4 kcal/mol. Some examples of hydrogen bonds with energy levels are F—H :F (38.6 kcal/mol), O—H :N (6.9 kcal/mol), O—H :O (5.0 kcal/mol), N—H :N (3.1 kcal/mol) and N—H :O (1.9 kcal/mol). See more in Perrin et al. “Strong” hydrogen bonds in chemistry and biology, Annual Review of Physical Chemistry, vol. 48, pages 511-544, 1997; Guthrie, “Short strong hydrogen bonds: can they explain enzymic catalysis?” Chemistry & Biology March 1996, 3:163-170.

In some embodiments, the components used in the present invention can form a strong hydrogen bond with the mutant polypeptide and/or its binding partner. These components tend to have an atom with a strong electronegativity. The atoms known to have the strongest electronegativity are F>O>Cl>N, in this order. Thus, the present invention preferably uses an organic compound that includes fluorine, a hydroxyl group or a carbonyl group, in forming the hydrogen bond. In one embodiment, organofluorines may be used in the present invention for forming a strong hydrogen bond.

In another embodiment, components capable of a hydrophobic interaction with a mutant polypeptide and/or its binding partner are employed. Such components include organic compounds with a hydrophobic group.

The hydrophobic interaction is a reversible attractive interaction between a hydrophobic compound or a hydrophobic region of a compound and another hydrophobic compound or hydrophobic region of the other compound. This type of interaction has been described in “Hydrophobic Interactions,” A. Ben-Nairn (1980), Plenum Press, New York.

Hydrophobic materials are repelled by water molecules because of their non-polar nature. When relatively nonpolar molecules or groups in aqueous solution associate with other nonpolar molecules or groups rather than with water, this is termed a “hydrophobic interaction.”

The mutant polypeptides and their binding partners typically include amino acids that are capable of hydrophobic interactions. These amino acids will typically be characterized by having at least one side chain with a nonpolar group that is capable of a hydrophobic interaction. Hydrophobic amino acids include, for example, alanine (Ala), isoleucine (Ile), leucine (Leu), phenylalanine (Phe), valine (Val), proline (Pro), glycine (Gly), to a lesser extent, methionine (Met), and tryptophan (Trp).

Components that are capable of hydrophobic interactions with a mutant polypeptide and/or its binding partner include organic compounds that are hydrophobic molecules or molecules containing at least one hydrophobic moiety. In some embodiments, these hydrophobic components may be hydrocarbons selected from aromatic hydrocarbons, substituted aromatic hydrocarbons, polyaromatic hydrocarbons, aromatic or non-aromatic heterocycles, cycloalkanes alkanes, alkenes, and alkynes. Hydrophobic groups may include aromatic groups, alkyl, cycloalkyl, alkenyl and alkynyl groups. The terms, “alkyl,” “alkenyl” and “alkynyl” as used herein refer to unsaturated aliphatic groups having one to thirty carbon atoms, including straight-chain alkenyl/alkynyl groups, branched-chain alkenyl/alkynyl groups, cycloalkenyl (alicyclic) groups, alkyl substituted cycloalkyl groups, and cycloalkyl substituted alkenyl/alkynyl groups. Such hydrocarbon moieties may also be substituted on one or more carbon atoms.

It may be understood that the strength of the hydrophobic interaction is based upon the available amount of “hydrophobes” that may interact one another. Thus, the hydrophobic interaction may be adjusted by, for example, increasing the amount of and/or “hydrophobic” nature of the hydrophobic moiety in the molecules involved in the hydrophobic interaction. For instance, a hydrophobic moiety, which in its original form may include a hydrocarbon chain, may be modified to increase its hydrophobicity (ability to increase the strength of hydrophobic interaction involved by the moiety) by having a hydrophobic side chain attach to one of the carbons of its carbon backbone. In a preferred embodiment , this may include the attachment of various polycyclic compounds, including for instance various steroidal compounds and/or their derivatives such as sterol type compounds, more particularly cholesterol. In general, the side chains may be linear chains, aromatic, aliphatic, cyclic, polycyclic, or any various other types of hydrophobic side chains as contemplated by those skilled in the art.

The type of components that are capable of van der Waals interactions with a mutant polypeptide and/or its binding partner are usually, but not always compounds with a polar moiety. As used herein, “van der Waals interactions” refer to attractions between atoms, moieties, molecules and surfaces that are caused by dipole-dipole interactions and/or correlations in the fluctuating polarizations of nearby atoms, moieties, molecules as a consequence of quantum dynamics.

The van der Waals interactions in the present invention are attractive forces between the mutant polypeptides or the binding partner and the component. The van der Waals interactions may arise from three sources. First, some molecules/moieties, although electrically neutral, may be permanent electric dipoles. Because of fixed distortion in the distribution of electron charge in the structure of some molecules/moieties, one side of a molecule/moiety is always somewhat positive and the opposite side somewhat negative. The tendency of such permanent dipoles to align with each other results in a net attractive force. This is interaction between two permanent dipoles (Keesom force).

Second, the presence of molecules that are permanent dipoles may temporarily distort the electron charge in other nearby polar or nonpolar molecules, thereby inducing further polarization. An additional attractive force results from the interaction of a permanent dipole with the neighboring induced dipole. This is an interaction between a permanent dipole and a corresponding induced dipole may be referred to as a Debye force. Third, even though no molecules involved are permanent dipoles (e.g., the organic liquid benzene), a force of attraction exists between molecules with two instantaneously induced dipoles in the molecules. This is interaction between two instantaneously induced dipoles may be referred to as a London dispersion force.

There are many amino acids in a mutant polypeptide and/or the binding partner that are capable of van der Waals interactions. These amino acids may have polar side chains, including glutamine (Gln), asparagine (Asn), histidine (His), serine (Ser), threonine (Thr), tyrosine (Tyr), cysteine (Cys), methionine (Met), tryptophan (Trp). These amino acids may also have a side chain with a non-polar group, including alanine (Ala), isoleucine (Ile), leucine (Leu), phenylalanine (Phe), valine (Val), proline (Pro), glycine (Gly).

The components that are capable of van der Waals interactions with a mutant polypeptide and/or its binding partner include polar or non-polar inorganic compounds that are soluble in the assay solution. The assay solution is generally an aqueous solution and thus these polar or non-polar inorganic compounds are preferably soluble in water. Preferred materials for van der Waals interactions are those that are polar such that they are capable of dipole-dipole interactions. For example AlF₃ has polar Al-F bonds and is soluble in water (about 0.67 g/100 ml water at 20° C.). HgCl₂ has polar Hg—Cl bonds and is soluble in water at 7.4 g/100 ml at 20° C. PrCl₂ has polar Pr—Cl bonds and is soluble in water at about 1 g/100 ml at 20° C.

Suitable polar compounds that are capable of van der Waals interactions include alcohols, thiols, ketones, amines, amides, esters, ethers, and aldehydes. Suitable examples of these compounds have been described above in relation to hydrogen bonding. Suitable non-polar compounds that are capable of van der Waals interactions include aromatic hydrocarbons, substituted aromatic hydrocarbons, polyaromatic hydrocarbons, aromatic or non-aromatic heterocycles, cycloalkanes, alkanes, alkenes, alkynes.

The hydrogen bonding components, hydrophobic components and Van der Waals components can be employed to influence binding of a mutant polypeptide and its binding partner in a number of ways. In one embodiment the hydrogen bonding, hydrophobic interaction and/or Van der Waals interaction may form a bridge between the mutant polypeptide and its binding partner. Such a bridge may bring the mutant polypeptide and binding partner into closer proximity to one another to facilitate binding and/or position the mutant polypeptide and/or binding partner relative to one another in a way that facilitates binding.

In another embodiment, the hydrogen bonding and/or hydrophobic interaction may increase the probability of the mutant polypeptide binding to its binding partner by, for example, by causing the polypeptides and binding partners to group or associate with one another in a manner which increases the binding probability. Thus, one or more of these interactions may be used alone or in combination to group the mutant polypeptides and binding partners closer together or to arrange the mutant polypeptides and binding partners in a manner that facilitates binding by, for example, causing the binding sites to be drawn closer together or causing the non-binding portions of the molecules to arrange further away from one another thereby allowing the binding sites to locate closer to one another.

In still another embodiment, the hydrogen bonding and/or hydrophobic interaction may influence the conformation of a mutant polypeptide and/or its binding partner to provide a conformation that is more conducive to the binding of the mutant polypeptide with its binding partner. Specifically, binding to or interacting with one or more of the amino acids of the mutant polypeptide and/or binding partner may cause one or more conformational shifts in the mutant polypeptide or binding partner that favors the mutant polypeptide/binding partner binding reaction.

The present invention conducts two pairs of assays, one to seek a decrease in activity for a mutant polypeptide in the assay at the normal physiological condition when compared to the parent polypeptide from which the mutant polypeptide was derived at said normal physiological condition, and a second assay to seek an increase in activity of the mutant polypeptide in the assay under the aberrant condition when compared to the parent polypeptide from which the mutant polypeptide was derived at said aberrant condition. The Examples described in WO 2017/078839 are illustrative of selecting conditionally active polypeptides than are more active at an aberrant pH at which activity is desired than at a normal physiological pH.

In one embodiment, the mutant polypeptides are subjected to an assay at the normal physiological condition and an assay under the aberrant condition. A conditionally active polypeptide is selected from the mutant polypeptides that have an increase in activity in the assay under the aberrant condition in comparison with the same activity of the mutant polypeptides in the assay under the normal physiological condition.

The condition used in the pairs of assays of the present invention may be selected from temperature, pH, osmotic pressure, osmolality, oxidative stress, electrolyte concentration and the concentration of any other component of the assay solution or media. Thus, a particular component of the assay media may be used at substantially the same concentration in both pairs of assays. In such case, the component is typically present for the purpose of simulating a particular environment in a human or animal such as serum, a tumor microenvironment, a synovial environment, a neural environment or any other environment which may be encountered at the point of administration, may be traversed by the administered treatment or may be encountered at the point of treatment. One important aspect of selecting one or more components that simulate these environments is that it may improve the results of the selection process carried out using the pairs of assays. For example, simulating a particular environment allows various effects of particular components of that environment on the mutant polypeptides to be evaluated in the selection process. Components of a particular environment may, for example, alter or bind with the mutant polypeptide, inhibit the activity of the mutant polypeptide, inactivate the mutant polypeptide, etc.

In some embodiments, one or more components of the assay solutions are preferably small molecules or ions, such as bisulfide, hydrogen sulfide, histidine, histamine, citrate, bicarbonate, lactate, and acetate. In one embodiment, the small molecule or ion component is preferably present in the assay solution at a concentration of from about 100 um to about 100 mM, or, more preferably from about 0.5 to about 50 mM, or from about 1 to about 10 mM.

The concentration of the component in the assay solutions may be the same or substantially the same as the concentration of the same component that is typically found in a naturally-occurring bodily fluid of a mammal, such as a human. This may be referred to as a normal physiological concentration of the component in the bodily fluid. In other embodiments, the concentration of a particular component in the assay solutions may be less than, or greater than the concentration of the same component that is typically found in a naturally-occurring bodily fluid of a mammal, such as a human.

In another embodiment, a component may be present at substantially different concentrations in each of the pairs of assays. In such case, the presence, absence or concentration of the component becomes the condition that is being assayed since it is the concentration of the component that is the condition that differentiates between the assay solutions for the assay under a normal physiological condition and the assay solution for the assay under an aberrant condition. Thus, the conditionally active polypeptide produced by this embodiment of the method of the present invention would be selected for an activity at least partially dependent on the concentration of the component.

In some embodiments, the component may be present in one pair of assay solutions but entirely absent from the other pair of assay solutions. For example, the concentration of lactate in the assay solution for the aberrant condition may be set to a level simulating a lactate concentration in the tumor microenvironment. Lactate may be absent from the pair of assay solutions for the normal physiological condition.

In one embodiment, the normal physiological condition is a first lactate concentration representative of a normal physiological condition and the aberrant condition is a second lactate concentration representative of an aberrant condition that exists in a particular location in the body.

In another example, glucose may be absent in the assay solution for the aberrant condition to simulate the absence of glucose that may be found in a tumor microenvironment, while glucose may be set to a level that simulates a blood plasma glucose concentration in the pair of assay solutions for the normal physiological condition. This feature may be used for preferential delivery of the conditionally active polypeptide to the location or environment without no or minimal activity in transit, and activation of the conditionally active polypeptide when it reaches the environment where the concentration of the component in the assay solution for the aberrant condition is present.

For example, a tumor microenvironment typically has both a lower glucose concentration and a higher lactate concentration in comparison with human serum. The normal physiological concentration of glucose is in the range of about 2.5 mM to about 10 mM in serum. On the other hand, the glucose concentration is typically very low in the range of 0.05 mM to 0.5 mM in the tumor microenvironment. In one embodiment, the assay solution for the assay under the normal physiological condition has a glucose concentration in the range of about 2.5 mM to about 10 mM and the assay solution for the assay under the aberrant condition has a glucose concentration in the range of about 0.05 mM to about 0.5 mM. The conditionally active polypeptide thus produced has a higher activity in a low glucose environment (in tumor microenvironment) than in a higher glucose environment (in normal tissues or blood). This conditionally active polypeptide will be functional in the tumor microenvironment but have a low activity in transit in the blood stream.

The normal physiological concentration of lactate in serum is in the range of about 1 mM to about 2 mM. On the other hand, lactate concentration is typically in the range of 10 mM to 20 mM in the tumor microenvironment. In one embodiment, the assay solution for the assay under the normal physiological condition has a lactate concentration in the range of about 1 mM to about 2 mM and the assay solution for the assay under the aberrant condition has a lactate concentration in the range of about 10 mM to about 20 mM. The conditionally active polypeptide thus produced has higher activity in a high lactate concentration environment (in tumor microenvironment) than in a lower lactate environment (in normal tissues or blood). This conditionally active polypeptide will thus be functional in the tumor microenvironment but have a low activity in transit in the blood stream.

Similarly, it is known that sore muscles have a higher (aberrant) concentration of lactate than normal. Thus, when seeking a mutant polypeptide that will be active in a sore muscle environment, the pair of assays at the aberrant condition can be conducted in the presence of a higher concentration of lactate to simulate the sore muscle environment, while the pair of assays at the normal physiological condition can be conducted with a lower concentration of, or in the absence of, lactate. In this manner, the mutant polypeptide can be selected for enhanced activity in a sore muscle environment with an increased lactate concentration. Such a conditionally active polypeptide may be useful as an anti-inflammatory agent, for example.

In another embodiment, two or more components may be used in both pairs of the assay solutions. In this type of assay, the conditionally active polypeptide may be selected using characteristics of both of the two types of assays described above. Alternatively, the selectivity of the conditionally active polypeptide can be increased using two or more components. For example, returning to the tumor microenvironment, the pair of assays at the aberrant condition can be conducted in assay media with both a high lactate concentration and a low glucose concentration while the corresponding pair of assays at the normal physiological condition can be conducted in an assay media with both a relatively lower lactate concentration and a relatively higher glucose concentration.

The present invention contemplates that each component selected from the inorganic compounds, ions, and organic molecules may be used alone or in combination to select a conditionally active polypeptide that is more active at one concentration of the component than at a different concentration of the same component.

Assays relying on different concentrations of one or more metabolites as the differentiating condition(s) between the normal environment (normal physiological condition) and the aberrant environment (aberrant condition) may be particularly suitable for selecting a conditionally active polypeptide that is more active in the tumor microenvironment than in blood plasma, because the tumor microenvironment typically has a significant number of metabolites that have different concentrations in comparison with the concentrations of the same metabolites in blood plasma.

Kinoshita et al., “Absolute Concentrations of Metabolites in Human Brain Tumors Using In Vitro Proton Magnetic Resonance Spectroscopy,” NMR IN BIOMEDICINE, vol. 10, pp.2-12, 1997, compared the metabolites in a normal brain and brain tumors. This group discovered that N-acetyl aspartate has a concentration of 5000-6000 μM in normal brain but the concentration is only 300-400 μM in glioblastoma, 1500-2000 μM in astrocytoma, and 600-1500 μM in anaplastic astrocytoma. Further, inositol has a concentration of 1500-2000 μM in a normal brain but the concentration is 2500-4000 μM in glioblastoma, 2700-4500 μM in astrocytoma, and 3800-5800 μM in anaplastic astrocytoma. Phosphorylethanolamine has a concentration of 900-1200 μM in a normal brain but the concentration is 2000-2800 μM in glioblastoma, 1170-1370 μM in astrocytoma, and 1500-2500 μM in anaplastic astrocytoma. Glycine has a concentration of 600-1100 μM in a normal brain but the concentration is 4500-5500 μM in glioblastoma, 750-1100 μM in astrocytoma, and 1900-3500 μM in anaplastic astrocytoma. Alanine has a concentration of 700-1150 μM in a normal brain but the concentration is 2900-3600 μM in glioblastoma, 800-1200 μM in astrocytoma, and 300-700 μM in anaplastic astrocytoma. These metabolites may also have different concentration in blood, for example, N-acetyl aspartate has a concentration of about 85000 μM in blood; inositol has a concentration of about 21700 μM in blood; glycine has a concentration of about 220-400 μM in blood; alanine has a concentration of about 220-300 μM in blood.

Therefore, these metabolites, including at least N-acetyl aspartate, inositol, glycine and alanine, may be used at different concentrations in the assay solutions to select conditionally active polypeptides that are active in brain tumors but not active in blood or normal brain tissue. For example, an assay solution with a concentration of 85000 μM of N-acetyl aspartate may be used for the pair of assays under a normal physiological condition and an assay solution with a concentration of 350 μM of N-acetyl aspartate may be used for the pair of assays under an aberrant condition to select conditionally active polypeptides that are active in the tumor microenvironment of glioblastoma, but not active or at least less active in blood or normal brain tissue.

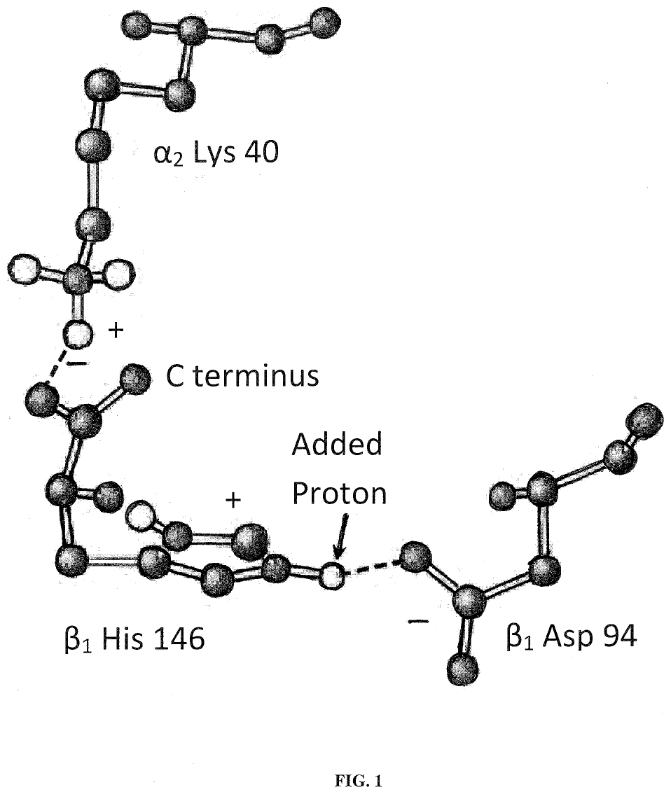

Mayers et al., “Elevated circulating branched chain amino acids are an early event in pancreatic adenocarcinoma development,” Nature Medicine, vol. 20, pp. 1193-1198, 2014, studied the concentrations of a variety of different metabolites including branched chain amino acids in prediagnostic blood plasma of pancreatic patients. It was found that in pancreatic tumor patients, there are several metabolites that are present in the bloodstream at different concentrations relative to the concentrations of the same metabolites in the blood of a human without pancreatic cancer. Mayers et al. supra also found that pancreatic cancer patients have significantly elevated branched amino acids in their blood plasma, in comparison with normal subjects. The branched amino acids that are present at elevated concentrations include isoleucine, leucine and valine (Table 1 of Mayers et al., supra). There are other metabolites shown in FIG. 1 of Mayers et al. supra that are present at significantly different concentrations in the blood plasma of pancreatic cancer patients than in normal healthy humans. These metabolites include at least acetylglycine, glycine, phenylalanine, tyrosine, 2-aminoadipate, taurodeoxycholate/taurochenodeoxycholate, aconitate, isocitrate, lactate, a-glycerophosphate and urate. Thus, based on the findings that certain metabolites are present at different concentrations in the blood plasma of pancreatic cancer patients and normal healthy patients, it can be predicted that the tumor microenvironment of pancreatic cancer will also have different concentrations for these metabolites than would be present in the pancreatic microenvironment of a healthy patient.

Thus, in one embodiment, one or more of these metabolites may be used in the assay solution for the normal physiological condition in amounts that approximate the concentrations of these metabolites in the blood plasma in a healthy individual (i.e., normal physiological concentrations of the metabolites). For example, the known normal physiological concentrations in blood plasma of a healthy individual are about 1.60±0.31 mg/dL for isoleucine, about 1.91±0.34 mg/dL for leucine, and about 2.83±0.34 mg/dL for valine. The assay solution for the normal physiological condition may have normal physiological concentrations within these ranges of one or more of these branched amino acids. The assay solution for the aberrant condition may have the same branched amino acids at concentrations that are about 5 fold, or about 10 fold, or about 20 fold, or about 50 fold, or about 70 fold, or about 100 fold, or about 150 fold, or about 200 fold, or about 500 fold higher than the normal physiological concentrations in a healthy individual of the corresponding branched amino acids. This would reflect the fact that the pancreatic tumor microenvironment would be expected to have significantly elevated concentrations of these branched amino acids based on the findings of Mayers et al. supra since the higher concentrations of these branched amino acids found in the blood plasma detected by Mayers et al. supra originate from the tumor microenvironment and are diluted in the blood stream. Similarly, the assay under the aberrant condition may reflect the concentrations of other metabolites in the blood of a pancreatic cancer patient even if the concentrations of particular metabolites are significantly lower in the cancer patient than in the normal individual. In this manner, the screening can simulate the actual environment and thereby ensure the highest activity mutants for that particular environment are selected.

In some other embodiments, the assay solution for the normal physiological condition may comprise one or more branched amino acids at concentrations simulating concentrations in the blood plasma of pancreatic cancer patients to simulate the actual blood plasma environment for these patients. In such embodiments, the assay solution for the aberrant condition may have the same branched amino acids at concentrations that are about 2 fold, or about 3 fold, or about 4 fold, or about 5 fold, or about 7 fold, or about 8 fold, or about 10 fold, or about 15 fold, or about 20 fold, or about 50 fold higher than the concentrations of the corresponding branched amino acids in the blood plasma of pancreatic cancer patients to reflect the fact that these higher concentrations are originating in the tumor microenvironment and the concentrations in the blood stream represent a dilution of the actual concentrations of the tumor microenvironment. Similarly, other metabolites may also have different concentrations in the assay solutions for the normal physiological condition and aberrant condition to reflect actual differences expected from the data collected for the blood stream. In some instances, a deficiency of a particular metabolite may be noted in the blood stream of a pancreatic patient in which case a concentration reflecting the measured concentration in the blood stream can be used in the assay for the normal physiological condition, and an even lower concentration can be used in the assay for the aberrant condition to account for the expectation that said metabolite is likely being consumed in the tumor microenvironment. The conditionally active polypeptides thus selected using the assay solutions will be more active in the pancreatic cancer microenvironment than in the blood plasma of pancreatic cancer patients.

In some embodiments, the entire blood plasma of pancreatic cancer patients may be used in the present invention. For example, in one embodiment, a simulation of one or more components of the blood plasma of pancreatic cancer patient may be used in the assay solutions for one or both of assays under the normal physiological condition and the aberrant condition. In an exemplary embodiment, the assay solution for the normal physiological condition has a pH in the range of 7.2-7.6 and with 30 wt. % of blood plasma of a pancreatic cancer patient added and the assay solution for the aberrant condition has a pH in the range of 6.2-6.8 and with 30 wt. % of blood plasma of pancreatic cancer patient added. In this embodiment, the blood plasma of the pancreatic cancer patient is present to both (1) ensure that the conditionally active polypeptide is not activated in the blood at pH 7.2-7.6, and (2) also ensure that the conditionally active polypeptide can be activated by the pH 5.5-7.2, 6-7, or 6.2-6.8 in the tumor microenvironment even in the presence of this composition of metabolites that is found in the blood of the pancreatic cancer patient. This will tailor the treatment for a pancreatic cancer patient.

In another exemplary embodiment, the assay solution for the normal physiological condition has a pH in the range of 7.2-7.6 and with 30 wt. % of blood plasma of pancreatic cancer patient added and the assay solution for the aberrant condition has a pH in the range of 5.5-7.2 or 6.2-6.8 and without any blood plasma of pancreatic cancer patient added.

The same component selected from the inorganic compounds, ions, and organic molecules may be used in each of the several types of assays discussed above. For example, in the case of lactate the lactate may be used at substantially the same concentration in the pairs of assay solutions for both normal physiological condition and aberrant condition. The normal physiological condition and aberrant condition will then differ in one or more other aspects, such as temperature, pH, concentration of another component, etc. In a different embodiment, the lactate may be used as one of the differentiating factors between the normal physiological condition and aberrant condition to reflect the fact that the lactate has a higher concentration in an aberrant tumor microenvironment than in a normal physiological condition (a non-tumor microenvironment).

In some embodiments, the two or more components are added at substantially the same concentration to both assay solutions for normal physiological condition and aberrant condition. For example, both citrate and bovine serum albumin (BSA) are added to the assay solutions. The citrate concentration may be about 80 μM and the BSA concentration may be about 10-20% in both assay solutions. More specifically, the assay solution for the pair of assays under the normal physiological condition may have a pH in the range of 7.2-7.6, with citrate at a concentration of about 80 μM and BSA at a concentration about 10-20%. The assay solution for the pair of assays under the aberrant condition may have a pH in the range of 6.2-6.8, with citrate at a concentration of about 80 μM and BSA at a concentration about 10-20%.

In one embodiment, serum may be added to both assay solutions for normal physiological condition and aberrant condition at substantially the same concentration. Because the serum has a large number of inorganic compounds, ions, organic molecules (including polypeptides), the assay solutions will have multiple and large number of components selected from inorganic compounds, ions, organic molecules presented at substantially the same concentrations between the two assay solutions. The assay solutions may have 5 to 30 vol. %, or 7 to 25 vol. %, or 10 to 20 vol. %, or 10 to 15 vol. %, of serum. In some other embodiments, the assay solutions for both normal physiological condition and aberrant condition are free of serum. The serum may be human serum, bovine serum, or serum from any other mammals. In some other embodiments, the assay solutions are free of serum.

The assay solutions for the normal physiological condition and aberrant condition may have different pHs. The pH of such assay solutions may be adjusted using CO₂ and O₂ levels in the buffer through use of bicarbonate.

In some other embodiments, at least one of the two or more components is added to the assay solutions for normal physiological condition and aberrant condition at different concentrations. For example, both lactate and bovine serum albumin (BSA) are added to the assay solutions. The lactate concentration may be different between the assay solutions for the normal physiological condition and aberrant condition, while the BSA may have the same concentration in both assay solutions. The lactate may have a concentration in the range of from 30 to 50 mg/dL in the assay solution for the aberrant condition and concentration in the range of from 8-15 mg/dL in the assay solution for the normal physiological condition. On the other hand, the BSA has the same concentration in both assay solutions, such as about 10-20%. The conditionally active polypeptide thus selected from using these assay solutions is more active at high lactate concentration at 30-50 mg/dL than at low lactate concentration at 8-15 mg/dL in the presence of BSA.

In some embodiments, the assay solutions may be designed for selecting conditionally active polypeptides with an activity dependent on two or more conditions. In one exemplary embodiment, the conditionally active polypeptide may have activity dependent on both pH and lactate. The assay solutions for selecting such a conditionally active polypeptide may be an assay solution for the normal physiological condition with pH at 7.2-7.6, lactate at a concentration in the range of from 8 to 15 mg/dL. The assay solution for the aberrant condition may have a pH at 6.2-6.8, lactate at a concentration in the range of from 30 to 50 mg/dL. Optionally the assay solutions for both normal physiological condition and aberrant condition may also comprise an ion to assist the binding between the mutant polypeptide and its binding partner, thus to increase the number of hits for candidate biologic active polypeptide.

In yet another exemplary embodiment, the conditionally active polypeptide may have activity dependent on pH, glucose and lactate. The assay solutions for selecting such a conditionally active polypeptide may be an assay solution for the normal physiological condition with pH at 7.2-7.6, glucose at a concentration in the range of 2.5-10 mM, lactate at a concentration in the range of from 8 to 15 mg/dL. The assay solution for the aberrant condition may be with pH at 6.2-6.8, glucose at a concentration in the range of 0.05 to 0.5 mM, lactate at a concentration in the range of from 30 to 50 mg/dL. Optionally the assay solutions for both normal physiological condition and aberrant condition may also comprise an ion to assist the binding between the mutant polypeptides and their binding partner, thus to increase the number of candidate biological active polypeptide binding to the binding partner at pH 6.2-6.8. The selected conditionally active polypeptide using such assay solutions is more active in an environment with pH 6.2-6.8, glucose concentration of 0.05 to 0.5 mM and lactate concentration of 30 to 50 mg/dL than in an environment with pH 7.2-7.6, glucose concentration of 2.5-10 mM and lactate concentration of 8 to 15 mg/dL.

The two or more components selected from inorganic compounds, ions, and organic molecules are for making an assay solution for the aberrant condition that simulates the environment at the location/site to which the selected conditionally active polypeptide will be delivered (i.e., targeted site). In some embodiments, at least three components presented in the environment at the targeted site may be added to the assay solution, or at least four components presented in the environment at the targeted site may be added to the assay solution, or at least five components presented in the environment at the targeted site may be added to the assay solution, or at least six components presented in the environment at the targeted site may be added to the assay solution.

In one embodiment, a fluid retrieved from the targeted site (where the conditionally active polypeptide will be more active) may be directly used as the assay solution for the assay under the aberrant condition. For example, synovial fluid may be retrieved from a subject, preferably from a subject with joint disease in need of treatment. The retrieved synovial fluid, optionally diluted, may be used as an assay solution in the pair of assays at the aberrant condition to select the conditionally active polypeptide. By using the retrieved synovial fluid, optionally diluted, as the assay solution for the assay under the aberrant condition, and an assay solution that simulates human blood plasma for the assay under the normal physiological condition, the conditionally active polypeptide (e.g., TNF-alpha) that is selected will be more active at the joint than at other locations or organs. For example, subjects with inflammatory joints (such as arthritis) may be treated with TNF-alpha. However, TNF-alpha typically has severe side effects of damaging other tissues and organs. A conditionally active TNF-alpha that is more active in the synovial fluid but not active or less active in blood will deliver the activity of TNF-alpha to the joints while reducing or potentially eliminating the side effects of the TNF-alpha on the rest of the body.

The development of conditionally active polypeptide that has an activity dependent on multiple conditions will result in improved selectivity of the conditionally active polypeptide to a target site in the body of a subject. Ideally, at other locations with only some of the conditions present the conditionally active polypeptide is not active or at least significantly less active. In one embodiment, the conditionally active polypeptide that is active at pH 6.2-6.8, glucose concentration of 0.05 to 0.5 mM and lactate concentration of 30 to 50 mg/dL can be specifically delivered to a tumor microenvironment because these conditions are all present in the tumor microenvironment. Other tissues or organs may have one or two of these conditions present but not all three, thus not be sufficient to fully activate the conditionally active polypeptide in the other tissues or organs. For example, the exercised muscle may have a low pH in the range of 6.2-6.8. However, it may not have another assayed condition. Thus the conditionally active polypeptide is not active or at least less active in the exercised muscle.

In some embodiments, steps may be taken to confirm that the activity of the conditionally active polypeptide is truly dependent on the conditions used to select the conditionally active polypeptide. For example, the conditionally active polypeptide is selected to be dependent on three conditions: pH 6.2-6.8, glucose concentration of 0.05 to 0.5 mM and lactate concentration of 30 to 50 mg/dL. The selected conditionally active polypeptide may then be tested at each of the three conditions individually and in environments with pairs of the three conditions to confirm that the conditionally active polypeptide is not active or less active in these test conditions or environments.

In some embodiments, certain components of serum may be purposely minimized or omitted from the assay media. For example, when screening antibodies, components of serum that bind with or adsorb antibodies can be minimized in or omitted from the assay media. Such bound antibodies may give false positives thereby including bound mutant antibodies that are not conditionally active but rather are merely bound to a component present in serum under a variety of different conditions. Thus, careful selection of assay components to minimize or omit components that can potentially bind with mutants in the assay can be used to reduce the number of non-functional mutants that may be inadvertently identified as positive for conditional activity due to binding to a component in the assay other than the desired binding partner. For example, in some embodiments where mutant polypeptides with a propensity to bond with components in human serum are being screened, BSA may be used in the assay solution in order to reduce or eliminate the possibility of false positives caused by mutant polypeptides bonding to components of human serum. Other similar replacements can also be made in particular cases to achieve the same goal.

In some embodiments, the assay conditions simulate the environment in the vicinity of a cell membrane such as inside, at or outside the cell membrane, or the environment in a joint. Some factors that may affect binding activities when screening in a cell membrane environment include expression of receptors, internalization, antibody drug complex (ADC) potency, etc.

The format of assays may be any suitable assays known to a person skilled in the art. Examples include ELISA, enzymatic activity assay, real tissue screening in vitro (organs, etc), tissue slides, whole animal, cell lines and use of 3D systems. For example, suitable cell-based assays are described in WO 2013/040445, tissue based assays are described in U.S. Pat. No. 7,993,271, whole animal based screening methods are described in US 2010/0263599, 3D system based screening methods are described in US 2011/0143960.

In some embodiments, the evolving step may produce mutant polypeptides that may simultaneously have other desired properties besides the conditionally active characteristics discussed above. Suitable other desired properties that may be evolved may include binding activity, expression, humanization, etc. Therefore, the present invention may be employed to produce a conditionally active polypeptide that also has an improvement in at least one or more of these other desired properties.

In some embodiments, the selected conditionally active polypeptide may be further mutated using one of the mutagenesis techniques disclosed herein in, for example, a second evolving step, to improve another property of the selected conditionally active polypeptide such as binding activity, expression, humanization, etc. After the second evolving step, the mutant polypeptides may be screened for both the conditional activity and the improved property.

In some embodiments, after evolving the parent polypeptide to produce mutant polypeptides, a first conditionally active polypeptide is selected, which exhibits an increase in first activity in the assay under an aberrant condition compared to the first activity in the normal physiological condition. The first conditionally active polypeptide may then be further subjected to one or more additional evolving, expressing and selecting steps to select at least a second conditionally active polypeptide that (1) exhibits an increase in a second activity in the assay under an aberrant condition compared to the second activity in an assay under the normal physiological condition, or (2) a larger ratio between the first activity at the aberrant condition and the first activity at the normal physiological condition, in comparison with the first conditionally active polypeptide and/or the parent polypeptide. The second conditionally active polypeptide may have both of the first activity and second activity higher under the aberrant condition in comparison with the respective activity under the normal physiological condition, as well as both of the first activity and second activity lower under the normal physiological condition in comparison with the parent polypeptide.

In certain embodiments, the present invention is aimed at producing conditionally active polypeptides with a large activity ratio of the activity at the aberrant condition and the activity at the normal physiological condition (e.g, a larger selectivity between the aberrant and normal physiological conditions). The ratio, or selectivity, of the activity at the aberrant condition and the activity at the normal physiological condition may be at least about 2:1, or at least about 3:1, or at least about 4:1, or at least about 5:1, or at least about 6:1, or at least about 7:1, or at least about 8:1, or at least about 9:1, or at least about 10:1, or at least about 11:1, or at least about 12:1, or at least about 13:1, or at least about 14:1, or at least about 15:1, or at least about 16:1, or at least about 17:1, or at least about 18:1, or at least about 19:1, or at least about 20:1, or at least about 30:1, or at least about 40:1, or at least about 50:1, or at least about 60:1, or at least about 70:1, or at least about 80:1, or at least about 90:1, or at least about 100:1.

In one embodiment, conditionally active polypeptide is an antibody, which may have a ratio between the activity at the aberrant condition and the activity at the normal physiological condition of at least about 5:1, or at least about 6:1, or at least about 7:1, or at least about 8:1, or at least about 9:1, or at least about 10:1, or at least about 15:1, or at least about 20:1, or at least about 40:1, or at least about 80:1. In one embodiment, the conditionally active polypeptide is used to target a tumor site where the conditionally active polypeptide is active at the tumor site (in tumor microenvironment) and significantly less active or inactive at a non-tumor site (normal physiological condition).

In one embodiment, the conditionally active polypeptide is an antibody that is intended to be conjugated with another agent such as those disclosed elsewhere herein. The conditionally active antibody may have a higher ratio of the activity at the aberrant condition and the activity at the normal physiological condition. For example, the conditionally active antibody that is to be conjugated with another agent may have a ratio of the activity at the aberrant condition to the activity at the normal physiological condition of at least about 10:1, or at least about 11:1, or at least about 12:1, or at least about 13:1, or at least about 14:1, or at least about 15:1, or at least about 16:1, or at least about 17:1, or at least about 18:1, or at least about 19:1, or at least about 20:1. This may be particularly important when the conjugated agent is, for example, toxic or radioactive, since such a conjugated agent is desirably concentrated at the disease or treatment site (where the aberrant condition is present).

### H. Confirming the pI of the Conditionally Active Polypeptide

In some embodiments, the pI of the selected conditionally active polypeptide is confirmed to be lower than the pI of the parent polypeptide using any of the techniques described above. For example, the pI of the conditionally active polypeptide may be lower than the pI of the parent polypeptide by at least 0.1, or at least 0.2, or at least 0.3, or at least 0.4, or at least 0.5, or at least 0.6, or at least 0.8, or at least 1.0, or at least 1.2, or at least 1.4, or at least 1.5, or at least 1.7, or at least 2.0, or at least 2.5, or at least 3.0, or at least 3.5, or at least 4.0, or at least 5.0 units lower than the pI of the parent polypeptide.

### I. Conditionally Active Polypeptides

In another aspect, the present invention provides conditionally active polypeptides that have a pI lower than the pI of the parent polypeptide. The conditionally active polypeptide has an increased activity at an aberrant condition that deviates from a normal physiological condition, compared to the same activity of the same mutant polypeptide at the corresponding normal physiological condition, and may optionally also have,

(a) an increased activity at an aberrant condition that deviates from a normal physiological condition, compared to the same activity of the parent polypeptide at the same aberrant condition,

(b) a decreased activity at a normal physiological condition compared with the same activity of the parent polypeptide at the same normal physiological condition, or

(c) a combination of (a) and (b) above.

The conditionally active polypeptides may be reversibly or irreversibly inactivated at the normal physiological condition, while being active at the aberrant condition. In some embodiments, these conditionally active polypeptides may have an activity at the aberrant condition that is the same or higher than the same activity of the parent polypeptide at the normal physiological condition.

Conditionally active polypeptides are particularly valuable for development of therapeutics that are active for a limited period of time within the host. This is particularly valuable where extended action of the therapeutic would be harmful to the host, but where limited activity is required to perform the desired therapy. Examples of beneficial applications include topical or systemic treatments, as well as localized treatments. One advantage of the conditionally active polypeptides is that they can enable use of higher dosages for therapeutic applications due to their ability to potentially reduce harmful side effects. Inactivation under the normal physiological condition can be used to reduce harmful side effects.

Inactivation at the normal physiological condition can be determined by a combination of the dose and the rate of inactivation of the polypeptide. This condition-based inactivation is especially important for enzyme therapeutics which may cause substantial negative side effects in a relatively short period of time.

The conditionally active polypeptide may also be reversibly or irreversibly activated or inactivated over time, or activated or inactivated only when it is located in a certain microenvironment in the body, including in specific organs in the body. Exemplary microenvironments may include, but are not limited to, microenvironments of a tumor, synovial fluid, and the bladder or the kidney.

In some embodiments, the conditionally active polypeptides are antibodies or antibody fragments against one or more target antigens as described herein.

The aberrant condition and normal physiological condition are as discussed above and may be a condition selected from temperature, pH, osmotic pressure, osmolality, oxidative stress, electrolyte concentration, as well as combinations of two or more such conditions. In some embodiments, the condition is pH and the conditionally active polypeptide has an activity that is pH dependent. Specifically, the conditionally active polypeptide has an increased activity at the aberrant pH compared with at the normal physiological pH.

In one aspect, the present invention relates to conditionally active polypeptides having pH-dependent activity in the presence of a species having a pKa within 0.5, 1, 1.5, 2, 2.5, 3 or 4 units of the pH at which the activity is desired. In another aspect, the present invention relates to conditionally active polypeptides having pH-dependent activity in the presence of a species having a pKa of from about 4 to about 10, or from about 4.5 to about 9.5 or from about 5 to about 9, or from about 5.5 to about 8, or from about 6.0 to about 7.0.

Species present in the assay media that have a significant influence on the activity of the conditionally active polypeptide tend to be species that have at least two ionization states: an uncharged or less charged state and a charged or more charged state. As a result, the pKa of the species that influences the activity of the conditionally active polypeptide can play a role in determining the degree of influence that the species will have on a particular activity of a polypeptide at a particular pH.

In another aspect, the present invention relates to conditionally active polypeptides having pH-dependent activity in the presence of a species selected from histidine, histamine, hydrogenated adenosine diphosphate, hydrogenated adenosine triphosphate, citrate, bicarbonate, acetate, lactate, bisulfide, hydrogen sulfide, ammonium, dihydrogen phosphate and any combination thereof. In some embodiments, the pH-dependent conditionally active polypeptides have a higher activity at a second pH than at a first, different pH, both activities being measured in an assay in the presence of these species. To determine pH-dependence of a conditionally active polypeptide the same activity of the polypeptide is assayed in the same assay media at two different pH values.

The ratio of the activity at the second pH to the same activity at the first pH in the same assay media is referred to as the selectivity of the pH-dependent conditionally active polypeptide. The pH-dependent conditionally active polypeptides have a selectivity of at least about 1.3, or at least about 1.5, or at least about 1.7, or at least about 2.0, or at least about 3.0, or at least about 4.0, or at least about 6.0, or at least about 8.0, or at least about 10.0, or at least about 20.0, or at least about 40.0, or at least about 60.0, or at least about 100.0.

It has been observed that frequently pH-dependent conditionally active polypeptides contain an increased number (or proportion) of charged amino acid residues in comparison to the amino acid residues of the parent polypeptide from which the conditionally active polypeptides are derived. There are three positively charged amino acid residues: lysine, arginine and histidine; and two negatively charged amino acid residues: aspartate and glutamate. In some embodiments, these charged amino acid residues are over-represented in pH-dependent conditionally active polypeptides in comparison with the parent polypeptides from which the pH-dependent conditionally active polypeptides are derived. As a result, the pH-dependent conditionally active polypeptides are more likely to interact with charged species in the assay media since the number of charged amino acid residues has been increased relative to the parent polypeptide. This, in turn, influences the activity of the conditionally active polypeptide.

It has also been observed that the pH-dependent conditionally active polypeptides typically have different activities in the presence of different species in the assay media. Species that have at least two ionization states: an uncharged or less charged state and a charged or more charged state may dissociate to a greater degree at a particular pH, dependent on their pKa value, to thereby increase the probability of interaction with charged amino acid residues present in the conditionally active polypeptide. This feature may be employed to enhance the selectivity and/or pH-dependent activity of the conditionally active polypeptide.

The nature of the charge(s) on the conditionally active polypeptide may be one factor used to determine suitable species for influencing the activity of the conditionally active polypeptide. In some embodiments, the conditionally active polypeptide may have more positively charged amino acid residues: lysine, arginine and histidine, in comparison with the parent polypeptide. Alternatively, the conditionally active polypeptide may have more negatively charged amino acid residues: aspartate and glutamate, in comparison with the parent polypeptide. The conditionally active polypeptide can thus be selected to have the desired level of interaction with a particular species present in the environment where the activity is desired and/or to have the desired level of interaction with a particular species present in the environment where a reduced activity is desired.

The location of the charged amino acid residues on the pH-dependent conditionally active polypeptide may also have an influence on the activity of the conditionally active polypeptide. For example, the proximity of charged amino acid residues to a binding site of the conditionally active polypeptide may influence the binding activity of the polypeptide.

In some embodiments, interaction of a charged environmental species with the conditionally active polypeptide may block or hinder the activity of the pH-dependent conditionally active polypeptide. For example, charged amino acids interacting with a charged environmental species may manifest allosteric effects on the binding site of the conditionally active polypeptide.

In other embodiments, the interaction of the charged environmental species with the conditionally active polypeptide may form salt bridges between different moieties on the polypeptide, especially the moieties that are charged or polarized. The formation of salt bridges is known to stabilize polypeptide structures (Donald, et al., “Salt Bridges: Geometrically Specific, Designable Interactions,” Proteins, 79(3): 898-915, 2011; Hendsch, et al., “Do salt bridges stabilize proteins? A continuum electrostatic analysis,” Protein Science, 3:211-226, 1994). The salt bridges can stabilize or fix the protein structure which normally undergoes constant minor structural variation called “breathing” (Parak, “Proteins in action: the physics of structural fluctuations and conformational changes,” Curr Opin Struct Biol., 13(5):552-557, 2003). The protein structure “breathing” is important for protein function and its binding because the structural fluctuation may permit the protein to efficiently recognize and bind to its partner (Karplus, et al., “Molecular dynamics and protein functions,” PNAS, vol. 102, pp. 6679-6685, 2015). By forming salt bridges, the binding site, especially the binding pocket, on the conditionally active polypeptide may be less accessible to its partner, possibly because the salt bridges may directly block the partner from accessing the binding site or may reduce protein structure “breathing.” Even with salt bridges remote from the binding site, the allosteric effect of the salt bridge may alter the conformation of the binding site to inhibit binding. Therefore, after the salt bridges stabilize (fix) the structure of the conditionally active polypeptide, the polypeptide may become less active in binding to its partner, leading to decreased activity.

One known example of a polypeptide having a structure that is stabilized by salt bridges is hemoglobin. Structural and chemical studies have revealed that at least two sets of chemical groups are responsible for the salt bridges in hemoglobin: the amino termini and the side chains of histidines β3146 and α122, which have pKa values near pH 7. In deoxyhemoglobin, the terminal carboxylate group of β146 forms a salt bridge with a lysine residue in the a subunit of the other αβ dimer. This interaction locks the side chain of histidine β146 in a position where it can participate in a salt bridge with negatively charged aspartate 94 in the same chain, provided that the imidazole group of the histidine residue is protonated (FIG. 1). At high pH, the side chain of histidine β146 is not protonated and the salt bridges do not form. As the pH drops, however, the side chain of histidine β146 becomes protonated and the salt bridge between histidine β146 and aspartate β94 forms, thereby stabilizing the quaternary structure of deoxyhemoglobin, leading to a greater tendency for oxygen to be released at actively metabolizing tissues (with lower pH). The hemoglobin shows a pH-dependent binding activity for oxygen as a result of which, at a low pH, the binding activity for oxygen is reduced because of the formation of salt bridges. On the other hand, at a high pH, the binding activity for oxygen is increased because of the absence of these salt bridges.

Similarly, ions such as bicarbonate ion may reduce the binding activity of the conditionally active polypeptide to its partner by forming salt bridges in the conditionally active polypeptides. For example, at a pH greater than the pKa of bicarbonate ion, bicarbonate ion is negatively charged. The negatively charged bicarbonate ion may form salt bridges between positively charged moieties or polarized moieties on the conditionally active polypeptide. These salt bridges may block or reduce the binding of the conditionally active polypeptide with its partner. At a pH lower than its pKa of 6.4, the bicarbonate ion is protonated and thus neutralized. The uncharged bicarbonate is not capable of forming salt bridges, and thus will not affect the binding of the conditionally active polypeptide with its partner in this manner In this scenario, the conditionally active polypeptide can have a higher binding activity with its partner at the pH below 6.4, the pKa of bicarbonate than at a higher pH above 6.4. In this example, the conditionally active polypeptide is conditionally active, i.e. exhibits a pH-dependent activity, in the presence of bicarbonate ion.

When a species such as bicarbonate is absent from the assay media, the conditionally active polypeptide may lose its conditional activity. This is likely due to the lack of salt bridges on the conditionally active polypeptide to stabilize (fix) the structure of the polypeptide. Thus, in the absence of bicarbonate, the binding partner may have a similar level of access to the binding site on the conditionally active polypeptide at any pH, thereby producing similar activity at any pH and eliminating the conditional activity.

In other embodiments, interactions between a small molecule or ion and the conditionally active polypeptide may alter the structure of the polypeptide in a manner that alters its activity. For example, the alteration in the structure may improve the binding affinity of the conditionally active polypeptide by altering a location, steric hindrance or binding energy for the binding site. In such cases, it may be desirable to select small molecules or ions that bind to the conditionally active polypeptide at the pH where activity is desired.

It is to be understood that, though the salt bridges (ionic bonds) are the strongest and most common manner for the compounds and ions to affect the activity of the conditionally active polypeptide, other interactions between such compounds and ions and the conditionally active polypeptide may also contribute to stabilize or fix the structure of the conditionally active polypeptide. Such other interactions include hydrogen bonding, hydrophobic interactions, and van der Waals interactions.

In some embodiments, to select a suitable compound or ion, the conditionally active polypeptide is compared with the parent polypeptide from which it is evolved to determine whether the conditionally active polypeptide has a higher proportion of negatively charged amino acid residues or positively charged amino acid residues. A compound or ion with a suitable charge at the pH at which activity is desired and the normal physiological pH, respectively, may then be chosen based on its size and pKa value, for use to influence the activity of the conditionally active polypeptide. For example, when the conditionally active polypeptide has a higher proportion of positively charged amino acid residues than the parent polypeptide, the suitable small molecule or ion should typically be negatively charged at the normal physiological pH to interact with the conditionally active polypeptide and should be neutral at the pH at which activity is desired. On the other hand, when the conditionally active polypeptide has a higher proportion of negatively charged amino acid residues than the parent polypeptide, the suitable small molecule or ion should typically be positively charged at the normal physiological pH to interact with the conditionally active polypeptide and neutral at the pH at which activity is desired.

In other embodiments, the activity of the conditionally active polypeptide is controlled by interaction of the small molecule or ion with a target polypeptide that is the binding partner of the conditionally active polypeptide. In this case the same principles as discussed above are also applicable except that the goal is to create interactions between the small molecule or ion and the target polypeptide. The target polypeptide can be, for example, an antigen of a conditionally active antibody, or a ligand of a conditionally active receptor.

A suitable small molecule or ion may be any inorganic or organic compound or ion that transits from an uncharged or less charged state at the pH at which activity is desired to a charged or more charged state at the normal physiological pH. Thus, the small molecule or ion should typically have a pKa between the pH at which activity is desired and the normal physiological pH. For example, bicarbonate has pKa at 6.4. Thus, at a such as pH 7.4, the negatively charged bicarbonate will bind to the charged amino acid residues in the conditionally active polypeptide and reduce the activity. On the other hand, at a lower pH such as pH 6.0, the less charged bicarbonate will not bind in the same quantity to the conditionally active polypeptide and thus allow a higher activity of the conditionally active polypeptide.

Bisulfide has a pKa 7.05. Thus, at a normal physiological pH such as pH 7.4, the more negatively charged bisulfide will bind to the positively charged amino acid residues in the conditionally active polypeptide and reduce its activity. On the other hand, at a lower pH such as pH 6.2-6.8, the less charged hydrogen sulfide/bisulfide will not bind at the same level to the conditionally active polypeptide and thus allow a higher activity of the conditionally active polypeptide.

Small molecules or ions with a pKa between the pH at which activity is desired and a normal physiological pH are preferred for use in the present invention. Preferred species are selected from bisulfide, hydrogen sulfide, histidine, histamine, citrate, bicarbonate, acetate, and lactate. Each of these small molecules or ions has a pKa between 6.2 and 7.0. Further, other small molecules such as tricine (pKa 8.05) and bicine (pKa 8.26) may also be used. Other suitable small molecules or ions may be found in textbooks such as the CRC Handbook of Chemistry and Physics, 96th Edition, by CRC press, 2015 and the Chemical Properties Handbook, McGraw-Hill Education, 1998, using the principles of the present application.

The concentration of the small molecules or ions in the assay media or environment is preferably at or near the physiological concentration of the small molecules or ions in a subject. For example, the physiological concentration of bicarbonate in human serum is in the range of 15 to 30 mM. Thus, the concentration of bicarbonate in the assay media may be from 10 mM to 40 mM, or from 15 mM to 30 mM, or from 20 mM to 25 mM, or about 20 mM. The concentration of bisulfide in the assay media may be from 3 to 500 nM, or from 5 to 200 nM, or from 10 to 100 nM, or from 10 to 50 nM.

In the invention, conditionally active polypeptides are selected and employed at concentrations whereby the normal physiological concentration of a particular species in an environment will have a significant effect on the activity of the conditionally active polypeptides in the pH range of interest. Thus, in many therapeutic treatments, it may be advantageous to have a low activity for the conditionally active polypeptide around pH 7.2-7.4 of blood or human serum to allow delivery of the therapeutic treatment via the bloodstream while minimizing or preventing the conditionally active polypeptide from activation. As a result, for such treatments it will be advantageous to select small molecules or ions having a pKa below pH 7.2-7.4 in order to ensure a sufficient amount of ionization of the small molecule at the bloodstream pH to have a significant effect on the activity of the conditionally active polypeptide. At the same time, the pKa of the small molecule or ion should be at or above the pH at which the activity of the conditionally active polypeptide is desired in order to ensure activation of the conditionally active polypeptide by protonation of the small molecule or ion to free up binding sites on the conditionally active polypeptide.

The small molecules or ions preferably have a low molecular weight and/or a relatively small conformation to ensure maximum access to small pockets on the target polypeptide or conditionally active polypeptide by minimizing steric hindrance. For this reason, the small molecules or ions typically have a molecular weight of less than 900 a.m.u., or more preferably less than 500 a.m.u. or more preferably less than 200 a.m.u. or even more preferably less than 100 a.m.u. For example, hydrogen sulfide, bisulfide and bicarbonate all have low molecular weights and small structures that provide access to pockets on the target polypeptide or conditionally active polypeptide, as shown in Examples 13 and 14 below.

The small molecule or ion may be present in the assays used to select for conditional activity or environments at substantially the same concentration, e.g. about 20 μM for bicarbonate. In some embodiments, the small molecule or ion may be present at different concentrations in different environments and thus it may be desirable to simulate this in the assays. For example, bisulfide has higher concentration in a tumor microenvironment than in human serum. Thus, the second assay may simulate a tumor microenvironment with an acidic pH and a higher concentration of bisulfide, while the first assay may simulate human serum with a neutral or slightly basic pH and a lower concentration of bisulfide. The acidic pH may be in the range from 6.0 to 6.8 while the neutral or slightly basic pH may be around 7.4. The higher concentration of bisulfide for the second assay simulating the tumor microenvironment may be 30 μM while the lower concentration of bisulfide for the first assay simulating human serum may be 10 μM or less, or 5μM.

In some embodiments, the conditionally active polypeptide is pH-dependent when two or more different small molecules and/or ions are present, for example, a combination of bicarbonate and histidine.

When the small molecule or ion is absent, the conditionally active polypeptide may lose its pH-dependency. Thus, in the absence of the small molecule or ion the conditionally active polypeptides may have similar activity between the aberrant pH at which activity is desired and the normal physiological pH.

In some embodiments, the aberrant pH at which activity is desired is an acidic pH while the normal physiological pH is a basic or neutral pH. For example, the aberrant pH may be a pH in the range of from about 5.5 to 7.2, or from about 6.0 to 7.0, or from about 6.2 to 6.8. The normal physiological pH may be a pH in the range of from greater than 7.2 to less than 7.6. Conditionally active polypeptides more active at an acidic pH and less active at a basic or neutral pH can target tumor microenvironment where the aberrant pH is acidic at from about 5.5 to 7.2, or from about 6.2 to 6.8.

In other embodiments, the aberrant pH at which activity is desired is a basic pH while the normal physiological pH is an acidic or neutral pH. For example, the aberrant pH at which the pH-dependent polypeptides are more active may be a basic pH of, for example, 7.6-7.9, such as in synovial fluid, (See Jebens et al., “On the viscosity and pH of synovial fluid and pH of blood,” Journal of Bone and Joint Surgery, vol. 41 B, pp. 388-400, 1959). The normal physiological pH may be the pH of blood of greater than 7.2 to below 7.6, at which the conditionally active polypeptides are less active. These conditionally active polypeptides may be suitable for targeting joint disease and joint inflammation.

In other embodiments, the conditionally active polypeptides may be designed to target the brain. There is a pH difference between the two sides of the blood brain barrier, with the pH on the brain side being about 0.2 pH units lower than the pH of blood or human serum. Thus, the aberrant pH of the brain at which the conditionally active polypeptides are more active may be about 7.0 to 7.2 (brain pH) while the normal physiological pH may be from greater than 7.2 to less than 7.6.

The conditionally active polypeptide may be an enzyme, a cytokine, a receptor especially a cellular receptor, a regulatory polypeptide, a soluble polypeptide, an antibody, or a hormone.

The conditionally active polypeptide may be a fragment of the parent polypeptide. For example, the conditionally active polypeptide may be an antibody fragment, a single chain antibody, a fragment of an enzyme, a fragment of a receptor, a fragment of a cytokine, or a fragment of a hormone. The antibody fragment may be an Fc fragment of antibody.

An Fc fragment may be used as the parent polypeptide for generating a conditionally active Fc fragment. The binding of the Fc fragment with the complement can be used to provide antibody-dependent cell mediated cytotoxicity. The aberrant pH may be acidic in the range of 5.5 to 7.2 or 6.2 to 6.8, such as the pH in the tumor microenvironment, while the normal physiological pH is in the range of greater than 7.2 to less than 7.6. The aberrant pH is different from the pH in the lysosomes where the pH is typically around 4.0. Further, the lysosomes are a location where the Fc fragment, like any other polypeptides, is targeted for degradation. There is no complement in the lysosomes and no cell mediated cytotoxicity to be caused through lysosomes.

The conditionally active polypeptide may have two functional domains with at least one, preferably both, of the functional domains having pH-dependent activity. These two functional domains may be evolved simultaneously and selection can be performed in a manner that identifies both functional domains in the same mutant polypeptide. Alternatively, the two functional domains may be independently evolved and selected. In this case, the two functional domains may be fused into a chimeric polypeptide.

In one aspect, the conditionally active polypeptide shows, in the presence of a factor such as a protein, an increased activity at the aberrant pH at which activity is desired in comparison with the parent polypeptide, and a decreased activity at the normal physiological pH in comparison with the parent polypeptide. The protein may be a protein present in blood, human serum or in a microenvironment of the body such as a tumor microenvironment, an inflamed area, synovial fluid, the brain, etc. One suitable protein may be albumin, particularly mammalian albumin, such as bovine albumin or human albumin

In one aspect, the protein such as albumin is present in the assay solutions used for screening and selecting the conditionally active polypeptide. In another aspect, the assay solutions with the protein such as albumin may also be used to test the activity of the selected conditionally active polypeptide under the same or different conditions.

### J. Engineering of Conditionally Active Polypeptides

The selected conditionally active polypeptides of the present invention may be further engineered using any of the methods described in WO 2017/078839 in the sections entitled “Engineering of conditionally active polypeptides,” “Engineering masked conditionally active polypeptide,” and “Engineering of conditionally active antibodies.” Further, the conditionally active polypeptides and the engineered conditionally active polypeptides of the present invention may be inserted into a viral particle that is an oncolytic virus, as described in WO 2017/078839.

### K. Production of the Conditionally Active Polypeptides

The selected conditionally active polypeptides and the engineered conditionally active polypeptides of the present invention may be produced for therapeutic use, prophylactic use, diagnostic use, research and related purposes, using the methods described in WO 2017/078839 in the section entitled “Production of the Conditionally Active polypeptides.”

### G. Pharmaceutical Compositions

Pharmaceutical composition comprising the conditionally active polypeptide, or the engineered conditionally active polypeptide, as well as the use of such pharmaceutical composition, are described in WO 2017/078839. The present invention extends to pharmaceutical compositions containing the conditionally active polypeptide or a further engineered version of the conditionally active polypeptide, which may be used in therapeutic, prophylactic and diagnostic applications.

### L. Uses of the Conditionally Active Polypeptides

The present invention also includes the use of the conditionally active polypeptides for therapeutic or prophylactic treatment of solid tumors, inflamed joints, or brain diseases or disorders.

Also included within the scope of the present invention are methods of treatment of solid tumors, inflamed joints, or brain diseases or disorders by administering the conditionally active polypeptides of the present invention to a patient in need of said treatment.

It must be noted that as used herein and in the appended claims, the singular forms “a”, “an”, and “the” include plural references unless the context clearly dictates otherwise. Furthermore, the terms “a” (or “an”), “one or more,” and “at least one” can be used interchangeably herein. The terms “comprising,” “including,” “having,” and “constructed from” can also be used interchangeably.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4. It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter,

All documents mentioned herein are hereby incorporated by reference in their entirety or alternatively to provide the disclosure for which they were specifically relied upon. The applicant(s) do not intend to dedicate any disclosed embodiments to the public, and to the extent any disclosed modifications or alterations may not literally fall within the scope of the claims, they are considered to be part hereof under the doctrine of equivalents.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meanings of the terms in which the appended claims are expressed.

1. A method of producing a conditionally active polypeptide from a parent polypeptide, comprising steps of:
(i) evolving the parent polypeptide by introducing one or more mutations into the parent polypeptide to produce one or more mutant polypeptides that have a pI that is the same as or lower than a pI of the parent polypeptide;
(ii) subjecting the one or more mutant polypeptides to a first assay under a normal physiological condition selected fromn the group consisting of pH, temperature, osmotic pressure, osmolality, oxidative stress, and electrolyte concentration to measure the activity of the one or more mutant polypeptides under the normal physiological condition and a second assay under an aberrant condition to measure the activity of the one or more mutant polypeptides under the aberrant condition, wherein the normal physiological condition and the aberrant condition are the same condition but having different values; and
(iii) selecting the conditionally active polypeptide from the one or more mutant polypeptides which exhibits an increased activity in the second assay compared to the same activity in the first assay.

2. The method of claim 1, wherein the conditionally active polypeptide has a pI that is lower than the pI of the parent polypeptide.

3. The method of claim 2, wherein the conditionally active polypeptide has a pI below 7.4.

4. The method of claim 2, wherein the one or more mutations comprise at least one amino acid substitution of a residue of an amino acid for a residue of an amino acid in the parent polypeptide that has a higher pI than a pI of the amino acid that is substituted into the parent polypeptide.

5. The method of claim 4, wherein the one or more mutations comprise 2 or more of said amino acid substitutions.

6. The method of claim 2, wherein the one or more mutations comprise at least one insertion of a residue of an amino acid that has a lower pI than the pI of the parent polypeptide.

7. (canceled)

8. The method of claim 2, wherein the one or more mutations comprise at least one deletion of a residue of an amino acid that has a higher pI than the pI of the parent polypeptide.

9. The method of claim 8, wherein the one or more mutations comprise 2 of said deletions.

10. The method of claim 2, wherein one or more of the mutations is located in a position exposed on the surface of the mutant polypeptide.

11. (canceled)

12. The method of claim 2, further comprising a step before step (ii) for confirming that at least 50% of the mutant polypeptides have a pI that is the same as or lower than the pI of the parent polypeptide.

13. The method of claim 12, further comprising a step of discarding mutant polypeptides that have a pI greater than the pI of the parent polypeptide prior to step (ii).

14. (canceled)

15. The method of claim 2, wherein the pI of the conditionally active polypeptide is lower than the pI of the parent polypeptide by at least 0.1 unit.

16. The method of claim 2, the parent polypeptide is selected from antibodies, enzymes, hormones, growth factors, cytokines, regulatory proteins, functional peptides, biosimilars, immunomodulators, receptors, and ligands.

17. The method of claim 2, wherein the parent polypeptide is an antibody selected from a full-length antibody, a single chain antibody, an antibody fragment, a heavy chain, a light chain, an Fab, and Fe domain.

18. (canceled)

19. The method of claim 2, wherein the parent polypeptide is an IgG antibody.

20. The method of claim 19, wherein the one or more mutations are in a variable region of the IgG antibody.

21. The method of claim 19, wherein the one or more mutations are in a constant region of the IgG antibody.

22. The method of claim 19, wherein the one or more mutations are in one or more complementarity determining regions of the IgG antibody.

23. (canceled)

24. The method of claim 2, wherein the condition is pH.

25. The method of claim 24, wherein the pH of the first assay is greater than 7.2 to less than 7.6 and the pH of the second assay is less than 7.2 or greater than 7.6.

26. The method of claim 3, wherein a ratio of the activity of the conditionally active polypeptide in the second assay to the same activity in the first assay is at least 1.3.

27. (canceled)

28. The method of claim 2, wherein both the first assay and second assay are performed in the presence of a molecule or ion selected from the group consisting of histidine, histamine, hydrogenated adenosine diphosphate, hydrogenated adenosine triphosphate, citrate, bicarbonate, acetate, lactate, bisulfide, hydrogen sulfide, ammonium, dihydrogen phosphate and any combination thereof.

29. The method of claim 2, wherein both the first assay and second assay are performed in the presence of a bicarbonate ion having a concentration in a range of from about 25 mM to about 200 mM.

30. The method of claim 2, wherein both the first assay and second assay are performed in the presence of a bisulfide ion having a concentration in a range of from 1 mM to 100 mM.

31. (canceled)

32. The method of claim 2, wherein the normal physiological condition is a normal physiological pH and the aberrant condition is an aberrant pH different from the normal physiological pH, both the first assay and second assay are performed in the presence of a molecule or ion that has a pKa between the normal physiological pH and the aberrant pH the pKa of the molecule or ion is up to 0.5 units away from the aberrant pH.

33. (canceled)

34. A conditionally active polypeptide derived from a parent polypeptide having a pI, said conditionally active polypeptide having:
a. a pI that is the same as or lower than the pI of the parent polypeptide; and
b. a ratio of at least 1.3 of an activity in a second assay at an aberrant condition to the same activity in a first assay at a normal physiological condition,
wherein the activity of the conditionally active polypeptide is measured in the presence of at least one molecule or ion having a molecular weight of less than 900 a.m.u., and
the condition is selected from the group consisting of pH, temperature, osmotic pressure, osmolality, oxidative stress, and electrolyte concentration.

35-48. (canceled)

49. A pharmaceutical composition comprising an effective amount of the conditionally active polypeptide of claim 34 and a pharmaceutically acceptable carrier.

50. (canceled)

51. A method of treatment of solid tumors, inflamed joints, or brain diseases or disorders comprising a step of administering the conditionally active polypeptide as claimed in claim 34 to a patient in need of said treatment.
